SEQUENCE LISTING (27 sequences)

SEQ ID NO: 1 - protein, 443 aa, synthetic construct
MALPVTALLLPLALLLHAARPEIVLTQSPATLSLSPGERATLSCRASQSISDYLHWYQQKPGQAPRLLIYFASQRATGIPARFSGSGSGTDFTLTISSLEPEDFAVYYCQQGHGFPRTFGGGTKVEIKGGGGSGGGGSGGGGSEVQLVESGGGLVQPGGSLRLSCAASGFTFSGYGMSWVRQAPGKGLEWVATITSGGTYTYYPDSVKGRFTISRDNAKNSLYLQMNSLRAEDTAVYYCARSLAGNAMDYWGQGTLVTVSSTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - DNA, 22 bp, synthetic construct
  source 1..22: mol_type = other DNA
catgttccaatatgattccacc

SEQ ID NO: 3 - DNA, 21 bp, synthetic construct
  source 1..21: mol_type = other DNA
gatgggatttccattgatgac

SEQ ID NO: 4 - DNA, 23 bp, synthetic construct
  source 1..23: mol_type = other DNA
atgatggcttattacagtggcaa

SEQ ID NO: 5 - DNA, 20 bp, synthetic construct
  source 1..20: mol_type = other DNA
gtcggagattcgtagctgga

SEQ ID NO: 6 - DNA, 23 bp, synthetic construct
  source 1..23: mol_type = other DNA
aaacaacctgaaccttccaaaga

SEQ ID NO: 7 - DNA, 22 bp, synthetic construct
  source 1..22: mol_type = other DNA
gcaagtctcctcattgaatcca

SEQ ID NO: 8 - DNA, 21 bp, synthetic construct
  source 1..21: mol_type = other DNA
acatcctgaagccaatcctga

SEQ ID NO: 9 - DNA, 20 bp, synthetic construct
  source 1..20: mol_type = other DNA
ctcctgtcgcatgtcactcc

SEQ ID NO: 10 - DNA, 23 bp, synthetic construct
  source 1..23: mol_type = other DNA
attcagggacctttcctattcgg

SEQ ID NO: 11 - DNA, 22 bp, synthetic construct
  source 1..22: mol_type = other DNA
ctcaccactattgaacttcccg

SEQ ID NO: 12 - DNA, 19 bp, synthetic construct
  source 1..19: mol_type = other DNA
cggcaacggagcttagagg

SEQ ID NO: 13 - DNA, 21 bp, synthetic construct
  source 1..21: mol_type = other DNA
ggcatagccttgaacgaacag

SEQ ID NO: 14 - DNA, 25 bp, synthetic construct
  source 1..25: mol_type = other DNA
gctcctgaggtatggaatagagtcc

SEQ ID NO: 15 - DNA, 20 bp, synthetic construct
  source 1..20: mol_type = other DNA
tatgtgtcatttgcgggggc

SEQ ID NO: 16 - DNA, 21 bp, synthetic construct
  source 1..21: mol_type = other DNA
ctctggcgtagagctatcact

SEQ ID NO: 17 - DNA, 20 bp, synthetic construct
  source 1..20: mol_type = other DNA
aggctgggttggtgaaaaca

SEQ ID NO: 18 - DNA, 23 bp, synthetic construct
  source 1..23: mol_type = other DNA
ctacaagggatcgggtttatgga

SEQ ID NO: 19 - DNA, 21 bp, synthetic construct
  source 1..21: mol_type = other DNA
ttggcattgcctagtagcgta

SEQ ID NO: 20 - DNA, 20 bp, synthetic construct
  source 1..20: mol_type = other DNA
acctgtccgagacctatggg

SEQ ID NO: 21 - DNA, 22 bp, synthetic construct
  source 1..22: mol_type = other DNA
cgtccagacgcataatgttgtc

SEQ ID NO: 22 - DNA, 21 bp, synthetic construct
  source 1..21: mol_type = other DNA
cagccagatgcaatcaatgcc

SEQ ID NO: 23 - DNA, 21 bp, synthetic construct
  source 1..21: mol_type = other DNA
cagccagatgcaatcaatgcc

SEQ ID NO: 24 - DNA, 22 bp, synthetic construct
  source 1..22: mol_type = other DNA
ccacaagatcgtgagaaaaccc

SEQ ID NO: 25 - DNA, 19 bp, synthetic construct
  source 1..19: mol_type = other DNA
cggtcctatgtgctcgtca

SEQ ID NO: 26 - DNA, 20 bp, synthetic construct
  source 1..20: mol_type = other DNA
ctgctgcactttggagtgat

SEQ ID NO: 27 - DNA, 20 bp, synthetic construct
  source 1..20: mol_type = other DNA
agatgatctgactgcctggg

(19) United States
(12) Patent Application Publication
(10) Pub. No.: US 2025/0205338 A1
(43) Pub. Date: Jun. 26, 2025

(54) COMPOSITIONS AND METHODS COMPRISING MODIFIED CHIMERIC ANTIGEN RECEPTOR (CAR) MACROPHAGES

(71) Applicant: Rutgers, The State University of New Jersey, New Brunswick, NJ (US)

(72) Inventors: Skylar Chuang, New Brunswick, NJ (US); KiBum Lee, New Brunswick, NJ (US)

(21) Appl. No.: 18/988,247

(22) Filed: Dec. 19, 2024

Related U.S. Application Data
(60) Provisional application No. 63/613,559, filed on Dec. 21, 2023.

(51) Int. Cl. A61K 40/42; A61K 40/17; A61K 40/31; A61K 47/68; A61K 47/69; C07K 16/28

(52) U.S. Cl. CPC A61K 40/421; A61K 40/17; A61K 40/31; A61K 47/6807; A61K 47/6849; A61K 47/6921; C07K 16/2803; C07K 16/289

(57) ABSTRACT

Described herein are compositions and methods for treating various indications in a subject including, but not limited to, atherosclerosis, cardiovascular diseases, inflammation, chronic inflammatory diseases, wounds, and spinal cord injuries. In some embodiments, the compositions and methods comprise a modified macrophage or monocyte comprising surface-expressed CD47-targeted chimeric antigen receptor (CAR) proteins and lipid-based particles conjugated to a surface of the modified macrophage or monocyte, wherein the lipid-based particles comprise a β-cyclodextrin (β-CD). In some embodiments, the lipid-based particles comprise lipid nanoparticles (LNPs).

### CROSS-REFERENCE TO RELATED APPLICATIONS

This application claims priority to U.S. Provisional Patent Application No. 63/613,559, filed on Dec. 21, 2023, which is incorporated by reference herein in its entirety.

### FEDERALLY SPONSORED RESEARCH

This invention was made with government support under grant DC016612 awarded by the National Institutes of Health (NIH). The government has certain rights in this invention.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, “210953-0007-US02_sequence_listing_xml_17 Dec. 2024.xml,” was created on Dec. 17, 2024, contains 27 sequences, has a file size of 28.0 kilobytes (28,672 bytes), and is incorporated by reference in its entirety into the specification.

### TECHNICAL FIELD

This disclosure generally relates to compositions and methods comprising modified chimeric antigen receptor (CAR) monocytes and macrophages.

### BACKGROUND

Programmed cell removal (PrCR) mediated by macrophages is a dynamic physiological process that plays a key role in immunosurveillance and maintaining tissue homeostasis. In particular, the clearance of dying apoptotic cells (ACs), or efferocytosis, is pivotal in the resolution phase of inflammation. Various diseases and conditions are characterized by inflammatory microenvironments that can affect the biology of both ACs and resident phagocytes, rendering efferocytosis dysfunctional.

Recent evidence implicates the role of impaired efferocytosis in the pathogenesis of atherosclerosis. Specifically, the upregulation of CD47, which is a “don't eat me” signal, on lesion foam cells significantly impedes efferocytosis when these cells undergo apoptosis. These ACs that are not readily cleared can then undergo secondary necrosis and contribute to chronic inflammation by increasing cellular oxidative stress and releasing pro-inflammatory intracellular factors. Moreover, cholesterol crystals (CC) that enrich lesion foam cells also contribute to inflammation through the NLRP3 inflammasome pathway. Collectively, these inflammatory hallmarks (i.e., necrotic cell debris and CC) cascade to further lesion expansion, plaque destabilization, and thrombosis. Hence, efficient removal of these detrimental hallmarks can help alleviate chronic inflammation and reduce the risks associated with cardiovascular disease. Although systemic administration of therapeutic agents such as CD47-blocking antibodies can improve efferocytosis and reduce plaque burden, these approaches can also lead to serious side effects such as anemia.

What is needed are therapeutic compositions and methods for effectively targeting ACs to enhance efferocytosis and reduce inflammation. Such compositions and methods would be useful in treating a variety of diseases and conditions characterized by increased inflammation, including atherosclerosis, cardiovascular diseases, chronic inflammatory diseases, wounds, and spinal cord injuries.

### SUMMARY

One embodiment described herein is a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD). In one aspect, the modified macrophage or monocyte is derived from a primary macrophage or monocyte, or wherein the modified macrophage or monocyte is derived from an induced pluripotent stem cell (iPSC). In another aspect, the CD47-targeted CAR proteins comprise an anti-CD47 single-chain variable fragment (scFv) comprising VL and VH; a CD8 hinge domain; a CD8 transmembrane domain; and a CD3ζ signaling domain. In another aspect, the CD47-targeted CAR proteins comprise an amino acid sequence having at least 90-99% identity to SEQ ID NO: 1. In another aspect, the CD47-targeted CAR proteins comprise an amino acid sequence of SEQ ID NO: 1. In another aspect, the lipid-based particles are lipid nanoparticles (LNPs) or liposomes. In another aspect, the lipid-based particles are LNPs comprising one or more of 1,2-dimyristoyl-sn-glycero-3-phosphocholine (DMPC), 1,2-dioleoyl-sn-glycero-3-phosphate (DOPA), cholesterol, or 1,2-distearoyl-sn-glycero-3-phosphoethanolamine-N-[amino(polyethylene glycol)-2000](DSPE-PEG(2000)). In another aspect, the β-CD is hydroxypropyl β-CD (HPβ-CD). In another aspect, the β-CD is modified with phenylboronic acid pinacol ester (PBAP). In another aspect, the lipid-based particles comprise a surface-conjugated anti-CD45 antibody that binds to CD45 expressed on the surface of the modified macrophage or monocyte. In another aspect, the lipid-based particles have a mean diameter of about 100 nm to about 350 nm. In another aspect, the lipid-based particles have a negative zeta potential of about −35 mV to about −50 mV. In another aspect, about 50 lipid-based particles to about 300 lipid-based particles are conjugated to the surface of the modified macrophage or monocyte. In another aspect, about 100 lipid-based particles to about 200 lipid-based particles are conjugated to the surface of the modified macrophage or monocyte. In another aspect, the modified macrophage or monocyte has enhanced phagocytosis and transmigration properties.

Another embodiment described herein is a pharmaceutical composition comprising any of the modified macrophages or monocytes described herein.

Another embodiment described herein is a method of treating atherosclerosis in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD). In one aspect, the β-CD is released from the lipid-based particles in response to elevated levels of reactive oxygen species (ROS) in the subject. In another aspect, the modified macrophage or monocyte reduces an amount of CD47-overexpressing apoptotic cells in the subject. In another aspect, the CD47-overexpressing apoptotic cells are apoptotic foam cells. In another aspect, the modified macrophage or monocyte reduces an amount of insoluble cholesterol in the subject. In another aspect, the modified macrophage or monocyte reduces inflammation in the subject.

Another embodiment described herein is a method of treating cardiovascular disease in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

Another embodiment described herein is a method of treating inflammation in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

Another embodiment described herein is a method of treating a chronic inflammatory disease in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

Another embodiment described herein is a method of treating a wound in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

Another embodiment described herein is a method of treating a spinal cord injury in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

This disclosure provides for other aspects and embodiments that will be apparent in light of the following detailed description and accompanying figures.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B show nanoimmuno-engineered CAR-Ms for potential atherosclerosis therapy. FIG. 1A (a1) shows a schematic illustration of CAR-transduced THP-1 monocytes anchored with β-CD LNPs. The inset shows a confocal micrograph of a CAR-monocyte carrying β-CD LNP backpacks. Scale bar is 5 μm. FIG. 1A (a2) shows a schematic depiction and transmission electron micrograph of a β-CD LNP. Scale bar is 100 nm. FIG. 1A (a3) shows a design of an anti-CD47 CAR construct. FIG. 1A (a4) shows that β-CD LNP-loaded CAR-Ms (CAR-M/β-CD LNP) can locate an inflamed lesion, traverse, and mediate the targeted clearance of lesion apoptotic cells (ACs) with elevated CD47 expression (CD47^{Hi} ACs). FIG. 1B shows that ingested cholesterol-rich materials (ACs and cholesterol crystals (CC)) can be scavenged by β-CD released from cell surface LNPs due to ROS-mediated degradation. Oxysterols converted from cholesterol facilitated by β-CD can activate the LXR signaling pathway and increase key downstream genes such as Abca1, Abcg1, and Mertk to promote cholesterol efflux and suppression of inflammation. Scale bars are 5 μm.

FIG. 2A-I show in vitro surface anchoring of β-CD LNPs onto monocytes. FIG. 2A shows a schematic illustration of β-CD LNPs loading onto THP-1 monocyte cells via CD45 targeting. FIG. 2B shows that size and zeta potential change before and after CD45 antibody conjugation. FIG. 2C shows loading efficiency of the β-CD LNPs based on a density of 1.0×10⁶ cells/mL. FIG. 2D shows confocal microscopy images of THP-1 cells (red) with β-CD LNP backpacks (green). Scale bar equals 20 μm. FIG. 2E shows that THP-1 monocytes were loaded with the indicated doses of β-CD LNPs and analyzed by flow cytometry. FIG. 2F-G show the effect of CD45 antibody targeting on cell-associated LNPs evaluated on day 0 and day 2 by flow cytometry (FIG. 2F) and confocal microscopy (FIG. 2G). Data represent mean±SEM; n=3; ***p<0.001 by unpaired two-tailed Student's t test. Scale bars equal 5 μm. FIG. 2H shows that surface β-CD LNPs containing DSPE-PEG-biotin were detected using a secondary reporter (AlexaFluor 647-streptavidin) by flow cytometry. FIG. 2I shows representative dot-plots of surface β-CD LNPs on THP-1 cells over 2 days analyzed by flow cytometry.

FIG. 3A-G show that β-CD LNP loading mediates protection and potentiates efferocytosis in macrophages. FIG. 3A shows a schematic depicting in vitro CC dissolution of THP-1 cells containing β-CD LNP backpacks. Data are mean±SEM; n=3; *p<0.05, ***p<0.001 by one-way ANOVA with Tukey post-hoc analysis; n.s. means no significance. FIG. 3B shows THP-1 cells with or without β-CD LNP backpacks challenged with various inflammatory mediators (LPS and IFN-γ; CC; oxLDL) to evaluate the protective effects of β-CD. FIG. 3C shows confocal microscopy images of THP-1 macrophages (red) examined 16 h after incubation with CC (green). Scale bar equals 20 μm. FIG. 3D shows canonical inflammasome genes in THP-1 macrophages after 3 h incubation with CC determined by RT-qPCR. FIG. 3E shows DCFDA fluorescence of THP-1 macrophages as a measure of intracellular ROS levels after 24 h of stimulation with LPS (100 ng/mL) and IFN-γ (50 ng/mL). FIG. 3F shows Nile red fluorescence ofTHP-1 to assess lipid content in THP-1 macrophages. FIG. 3G shows gene expression changes in THP-1 macrophages with or without β-CD LNP backpacks incubated with apoptotic Jurkat cells (ACs) determined by RT-qPCR. Data represent mean±SEM; n=3; *p<0.05, **p<0.01, ***p<0.001 by unpaired two-tailed Student's t test.

FIG. 4A-I show CAR-M engineering and phagocytosis activity. FIG. 4A shows a schematic diagram illustrating the process generating CAR-Ms from THP-1 cells. FIG. 4B shows a lentiviral construct used for CAR transduction. FIG. 4C shows verification of surface expression of anti-CD47 CARs in CAR-Ms. FIG. 4D shows that the anti-CD47 CAR represents a chimeric switch receptor (CSR) that reverses existing inhibitory signals mediated by the SIRPa-CD47 axis to enhance phagocytosis of CD47^{Hi} ACs. FIG. 4E shows confocal micrographs depicting phagocytosis of CD47^{Hi} ACs in control macrophages, macrophages with CD47-blocked ACs, and CAR-Ms after 1 h incubation at 37° C. Scale bar equals 20 μm. FIG. 4F shows representative fluorescence microscopy images of control macrophages and CAR-Ms incubated with ACs for 2 h at 37° C. The cells were washed three times prior to imaging. Scale bar equals 100 μm. FIG. 4G-I show quantitative analysis of partial (FIG. 4G), full (FIG. 4H), and total (FIG. 4I) engulfment of CD47^{Hi} ACs using CellTagging after 2 h co-culture at 37° C. Data are mean±SEM; n=5; *p<0.05, **p<0.01 by unpaired two-tailed Student's t test.

FIG. 5A-H show CAR-M phagocytosis under inflammatory condition. FIG. 5A shows a schematic demonstrating that, in addition to increasing CD47 expression in lesion cells, chronic inflammation also reduces the capacity of macrophages to clear ACs via ectodomain shedding. Efferocytic receptors (e.g., MerTK) can be cleaved by the metalloproteinase ADAM17, leading to reduced efferocytosis overall. FIG. 5B shows representative fluorescence micrographs depicting phagocytosis of standard ACs (without inducing CD47 elevation) by M1 control macrophages or CAR-Ms after 2 h incubation. Arrowheads indicate fully internalized ACs. Scale bar equals 100 μm. FIG. 5C-E show the quantification of partial (FIG. 5C), full (FIG. 5D), and total (FIG. 5E) internalization of standard ACs by either M1 control macrophages or CAR-Ms by CellTagging.

FIG. 5F shows quantitative analysis of the phagocytosis of standard ACs by control macrophages or CAR-Ms pretreated with either TNF-α alone or TNF-α and LPS. Data are mean±SEM; n=3; *p<0.05, **p<0.01 by unpaired two-tailed Student's t test. FIG. 5G shows combined M1 macrophages with CD47^{Hi} ACs co-culture to simulate the phagocytosis in the atherosclerotic lesion environment. Scale bar equals 100 μm. FIG. 5H shows suppression of TNF-α expression in macrophages upon efferocytosis with CD47^{Hi} ACs characterized by RT-qPCR. Data are mean±SEM; n=3; *p<0.05, **p<0.01 by one-way ANOVA with Tukey post-hoc analysis.

FIG. 6A-J show an assessment of CAR-M phagocytosis combined with β-CD LNPs. FIG. 6A shows the various conditions selected to evaluate macrophage phagocytosis of CD47^{Hi} ACs.

FIG. 6B-D show quantitative analysis via CellTagging of partial (FIG. 6B), full (FIG. 6C), and total (FIG. 6D) engulfment after 1 h incubation of macrophages with CD47^{Hi} ACs at 37° C. FIG. 6E shows that the activation of LXR signaling targets Abca1 and Abcg1, as well as efferocytosis targets IL-10 and Mertk in control macrophages with β-CD LNPs or CAR-Ms with β-CD LNPs after co-culture with CD47^{Hi} ACs for 2 h 37° C. FIG. 6F shows time lapse fluorescence imaging of CAR-Ms with CD47^{Hi} ACs for 1 h. Scale bar equals 45 μm. FIG. 6G (top) shows a 3D rendering of the microfabricated vessel-on-a-chip device constructed using photolithography. FIG. 6G (bottom) shows HUVECs were seeded in the top channel and visualized by microscopy. Scale bars equal 100 μm. FIG. 6H shows the experimental timeline for HUVEC activation and CAR-M co-culture. FIG. 6I shows fluorescence microscopy images examining the adherence of control THP-1 cells and CAR-Ms to TNF-α-activated HUVECs in the device. Scale bars equal 20 μm.

FIG. 6J shows confocal microscopy images and quantitation of CAR-Ms in the GeIMA hydrogel layer indicating transendothelial migration. The CAR-Ms were fluorescently labeled with β-CD LNPs (red). Scale bar equals 100 μm. Data are mean±SEM; n=3, **p<0.01, ***p<0.001 by one-way ANOVA with Tukey post-hoc analysis; n.s. means not significant.

FIG. 7A-C show the characterization of PBAP-modified HP-pCD. FIG. 7A shows a schematic of the PBAP modification process. FIG. 7B shows an FT-IR spectra of PBAP-modified HP-βCD. FIG. 7C shows a ¹H-NMR spectra of PBAP-modified HP-βCD.

FIG. 8A-F show the preparation and characterization of β-CD LNPs. FIG. 8A shows a schematic illustration of the nanoprecipitation process for the synthesis of β-CD LNPs. FIG. 8B shows a representative TEM image of β-CD LNPs stained with 2% uranyl acetate. Scale bar equals 50 nm. FIG. 8C shows an XPS spectrum of characteristic elements found in β-CD LNPs.

FIG. 8D shows cell viability measured by PrestoBlue in THP-1 macrophages and HUVECs. FIG. 8E shows dynamic light scattering (DLS) data showing the size distribution of β-CD LNPs to be between 50-1000 nm. FIG. 8F shows polydispersity index (PDI) data representing the size uniformity of β-CD LNPs.

FIG. 9A-C show ROS-mediated release and antioxidant activity of β-CD LNPs. FIG. 9A shows the degradation of LNPs and the release of β-CD at different concentrations of H₂O₂. FIG. 9B shows Rhodamine B absorbance after UV light irradiation in the presence of different concentrations of β-CD LNPs. FIG. 9C shows the percentage of intracellular ROS reduced in THP-1 macrophages treated with different concentrations of β-CD LNPs. Data are mean±SEM; n=3; *p<0.05, **p<0.01, ***p<0.001 by one-way ANOVA followed by a Tukey post-hoc test; n.s. means no significance.

FIG. 10A-D show the CC scavenging and anti-inflammatory effects of β-CD LNPs. FIG. 10A shows an assessment of the CC-dissolving capability of β-CD LNPs in the presence of fixed H₂O₂ at increasing concentrations. The samples were filtered, and supernatant (filtrate) collected for fluorescence analysis. The crystalline fractions were obtained by incubating the filters with hot methanol and fluorescence was analyzed. FIG. 10B (top) shows a timeline of treatment with β-CD LNPs in THP-1 macrophages. THP-1 macrophages were differentiated for 48 h before incubation with CC for 3 h. The cells were subsequently washed and incubated for an additional 18 h prior to imaging shown in FIG. 10B (bottom). The arrowheads indicate intracellular CC after 18 h. Scale bar equals 100 μm. FIG. 10C shows quantitative assessment of intracellular CC after 3-CD LN P treatment. FIG. 10D shows suppression of inflammation and upregulation of LXR target genes after treatment with β-CD LNPs characterized by RT-qPCR. Data are mean±SEM; n=3; *p<0.05, **p<0.01, ***p<0.001 by ANOVA followed by a Tukey post-hoc test; n.s. means no significance.

FIG. 11A-D show phagocytosis of CD47-coated beads and CD47^{Hi} ACs. FIG. 11A shows a schematic illustration of a bead phagocytosis assay and confocal microscopy images of the phagocytosis of lipid-coated beads containing CD47 by control macrophages (transduced with blank virus) and CAR-Ms. The beads were used to mimic apoptotic bodies. FIG. 11B shows Z-stack imaging of a CAR-M cell with internalized CD47 beads. FIG. 11C shows quantification of beads in control macrophages and CAR-Ms. FIG. 11D shows phagocytosis of MCF-7 derived CD47^{Hi} ACs subjected to TNF-α for 2 d. Confocal microscopy images depicted both control macrophages (transduced with blank virus) and CAR-Ms ingesting target ACs.

FIG. 12A-C show TNF-α-induced upregulation of CD47 in apoptotic MCF-7 cells. FIG. 12A shows fluorescence microscopy of MCF-7 cells treated with different amounts of TNF-α (0, 50, and 100 ng/mL) for 2 d under both healthy (no STS) or apoptotic (+STS) conditions. Scale bars equal 50 μm. FIG. 12B shows flow cytometry of viable or apoptotic MCF-7 cells treated with or without TNF-α for 2 d. FIG. 12C shows confocal microscopy of viable or apoptotic MCF-7 cells treated with or without TNF-α for 2 d. Scale bar equals 20 μm.

FIG. 13 shows the workflow employed for the image-based quantification of phagocytosis by CellTagging. A schematic of the major steps involved in automated quantification of fluorescent signal colocalization is shown. Objects are detected from the background and the picture is binalized using the Otsu method. Objects are detected based on the size threshold set for grouping pixels with an object together. The cell debris and any aggregates are then filtered out of the green fluorescent channel before the fluorescent signal of the red channel overlapping with the green fluorescent signal is quantified, and a final percentage of cells with co-localization of fluorescent signals is provided.

FIG. 14A-F show CAR-M transmigration in AC-embedded GeIMA hydrogels. FIG. 14A shows the major steps that are involved in atherosclerotic lesion development and examined in a microfabricated vessel model. FIG. 14B shows an assessment of control macrophage, CAR-M, and CD47 blockade on HUVEC viability by flow cytometry. FIG. 14C shows representative fluorescence microscopy images of HUVECs with or without TNF-α treatment. FIG. 14D shows dose- and time-dependent assessments of CD54 upregulation in HUVECs after TNF-α treatment. FIG. 14E shows representative fluorescence microscopy images and quantitation of CAR-M or CAR-M/β-CD LNP transmigration in GeIMA hydrogels in vitro. Arrows indicate the GFP+ CAR-Ms. FIG. 14F shows a 3D rendering of the designed vessel-on-a-chip device and its dimensions. Data are mean±SEM; n=3, **p<0.01, ***p<0.001 by one-way ANOVA with Tukey post-hoc analysis; n.s. means not significant.

Before any embodiments of this disclosure are explained in detail, it is to be understood that the disclosure is not limited in its application to the details of construction and the arrangement of components set forth in the following description or illustrated in the accompanying figures. Also, it is to be understood that the phraseology and terminology used herein is for the purpose of description and should not be regarded as limiting.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30° C., including all integers and endpoints within the specified range. In another aspect, “room temperature” refers a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ˜20° C., ˜22° C., ˜25° C., or ˜27° C., at standard atmospheric pressure.

As used herein, the terms “amino acid,” “gene,” “nucleic acid,” “nucleotide,” “polynucleotide,” “oligonucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein. Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine, hypoxanthine, isocytosine and isoguanine. Nucleic acids may be obtained by chemical synthesis methods or by recombinant methods.

As used herein, “variants” can include, but are not limited to, those that include conservative amino acid (AA) substitution, SNP variants, degenerate variants, and biologically active portions of a gene. A “degenerate variant” as used herein refers to a variant that has a mutated nucleotide sequence, but still encodes the same polypeptide due to the redundancy of the genetic code. There are 20 naturally occurring amino acids; however, some of these share similar characteristics. For example, leucine and isoleucine are both aliphatic, branched, and hydrophobic. Similarly, aspartic acid and glutamic acid are both small and negatively charged. Conservative substitutions in proteins often have a smaller effect on function than non-conservative mutations. Although there are many ways to classify amino acids, they are often sorted into six main groups on the basis of their structure and the general chemical characteristics of their R groups. A mutation among the same class of amino acids is considered a conservative amino acid substitution.

The term “functional” when used in conjunction with “variant” or “fragment” refers to an entity or molecule which possess a biological activity that is substantially similar to a biological activity of the entity or molecule of which it is a variant or fragment thereof. In accordance with the present disclosure, a modified macrophage or monocyte, or a CAR protein or polypeptide, may be modified, for example, to facilitate or improve activity, stability, identification, expression, isolation, storage, and/or administration, so long as such modifications do not reduce its function to an unacceptable level.

As used herein, “substantial identity” of polynucleotide sequences means that a polynucleotide comprises a sequence that has at least 25% sequence identity compared to a reference sequence as determined using programs known in the art (e.g., Basic Local Alignment Search Tool (BLAST)). In preferred embodiments, percent identity can be any integer from 25% to 100%. More preferred embodiments include polynucleotide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. These values can be appropriately adjusted to determine corresponding identity of proteins encoded by two nucleotide sequences by taking into account codon degeneracy, amino acid similarity, reading frame positioning, and the like. Accordingly, polynucleotides of the present disclosure encoding a protein or polypeptide of the present disclosure include nucleic acid sequences that have substantial identity to the nucleic acid sequences that encode the proteins or polypeptides of the present disclosure. Polynucleotides encoding a polypeptide comprising an amino acid sequence that has at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference polypeptide sequence are also preferred.

As used herein, “substantial identity” of amino acid sequences (and of polypeptides having these amino acid sequences) means that an amino acid sequence comprises a sequence that has at least 25% sequence identity compared to a reference sequence as determined using programs known in the art (e.g., BLAST). In preferred embodiments, percent identity can be any integer from 25% to 100%. More preferred embodiments include amino acid or polypeptide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. Polypeptides that are “substantially identical” share amino acid sequences except that residue positions which are not identical may differ by one or more conservative amino acid changes, as described above. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acid substitution groups include valine-leucine-isoleucine, phenylalanine-tyrosine, lysine-arginine, alanine-valine, aspartic acid-glutamic acid, and asparagine-glutamine. Accordingly, polypeptides or proteins, encoded by the polynucleotides of the present disclosure, include amino acid sequences that have substantial identity to the amino acid sequences of the reference polypeptide sequences.

As used herein, the terms “active ingredient” or “active pharmaceutical ingredient” refer to a pharmaceutical agent, active ingredient, compound, cell, or substance, compositions, or mixtures thereof, that provide a pharmacological, therapeutic, often beneficial, effect. In some embodiments, disclosed compositions may further comprise one or more pharmaceutically acceptable carriers or excipients. Example carriers may include, but are not limited to, liposomes, polymeric micelles, microspheres, microparticles, dendrimers, or nanoparticles.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “administration” or “administering” refers to providing, contacting, and/or delivery of an action, agent, composition, or cell(s) by any appropriate route to achieve a desired effect. In some embodiments, the term “administering” may also refer to the placement of an agent or a composition as disclosed herein into a subject by a method or route which results in at least partial localization of the agents or composition at a desired site. “Route of administration” may refer to any administration pathway known in the art, including but not limited to oral, intravenous (IV), topical, aerosol, nasal, via inhalation, anal, intra-anal, peri-anal, transmucosal, transdermal, parenteral, enteral, or local. “Parenteral” refers to a route of administration that is generally associated with injection, including intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous (IV), intraarterial, subarachnoid, subcapsular, subcutaneous, transmucosal, or transtracheal. Via the parenteral route, the agent or composition may be in the form of solutions or suspensions for IV infusion or IV injection, or as lyophilized powders. Via the enteral route, the agent or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the agent or composition can be in the form of aerosol, lotion, cream, gel, ointment, suspensions, solutions, or emulsions. In one embodiment, the agent or composition may be provided in a powder form and mixed with a liquid, such as water, to form a beverage. In accordance with the present disclosure, “administering” can be self-administering. For example, it is considered “administering” when a subject consumes a composition as disclosed herein.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and the type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human. The methods and compositions disclosed herein can be used on a sample either in vitro (for example, on isolated cells or tissues) or in vivo in a subject (i.e., a living organism, such as a human patient). In some embodiments, the subject comprises a human who is undergoing treatment using a composition and/or method as prescribed herein.

As used herein, a subject is “in need of treatment” if such subject would benefit biologically, medically, or in quality of life from such treatment. A subject in need of treatment does not necessarily present symptoms, particularly in the case of preventative or prophylaxis treatments. In some embodiments of the present disclosure, a subject is in need of treatment if the subject is suffering from, or at risk of suffering from, one or more of atherosclerosis, cardiovascular disease, inflammation, chronic inflammatory disease, a wound, or a spinal cord injury.

It will be appreciated that appropriate dosages of the active cells and compositions disclosed herein can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular cell or composition, the route of administration, the time of administration, the rate of excretion of the composition, the duration of the treatment, other drugs, compounds, and/or materials used in combination, and the age, sex, weight, condition, general health, and prior medical history of the patient. The amount of cell or composition and the route of administration will ultimately be at the discretion of a trained physician, although generally, the dosage will be to achieve local concentrations at the site of action which achieve the desired effect without causing substantial harmful or deleterious side-effects. The actual dosage can also depend on the determined experimental effectiveness of the specific cell or composition that is administered. For example, the dosage may be determined based on in vitro responsiveness of relevant cultured cells, or in vivo responses observed in appropriate animal models or human studies.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In some embodiments of the present disclosure, a subject may be administered a single dose of the disclosed pharmaceutical compositions. In other embodiments, the subject may be administered a plurality of doses of the disclosed pharmaceutical compositions over a period of time. For example, in various nonlimiting embodiments, a pharmaceutical composition comprising a modified macrophage or monocyte as described herein may be administered to a subject once a day (SID/QD), twice a day (BID), three times a day (TID), four times a day (QID), or more, so as to administer a therapeutically effective amount of the pharmaceutical composition to the subject, where the therapeutically effective amount is any one or more of the doses described herein. In some embodiments, a pharmaceutical composition as described herein is administered to a subject 1-3 times per day, 1-7 times per week, 1-9 times per month, 1-12 times per year, or more. In other embodiments, a pharmaceutical composition as described herein is administered for about 1-10 days, 10-20 days, 20-30 days, 30-40 days, 40-50 days, 50-60 days, 60-70 days, 70-80 days, 80-90 days, 90-100 days, 1-6 months, 6-12 months, 1-5 years, or more. In various embodiments, a pharmaceutical composition as described herein is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, 900-1000 mg/kg, or a combination thereof. The actual dosing regimen can depend upon many factors, including but not limited to the judgment of a trained physician, the overall condition of the subject, and the specific disease or condition of the subject. The actual dosage can also depend on the determined experimental effectiveness of the specific pharmaceutical composition that is administered. For example, the dosage may be determined based on in vitro responsiveness of relevant cultured cells, or in vivo responses observed in appropriate animal models or human studies.

For example, a therapeutically effective amount of a pharmaceutical composition disclosed herein may be about 0.1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

As used herein, the term “endogenous” refers to any material from or produced inside an organism, cell, tissue, or system.

As used herein, the term “exogenous” refers to any material introduced from or produced outside an organism, cell, tissue, or system.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic manner. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifested.

As used herein, “sample” or “target sample” refers to any sample in which the presence and/or level of a target analyte or target biomarker is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological or bodily fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, “target analyte,” “target biomarker,” “target antigen,” or “target cell” refers to a substance that is associated with a biological state or a biological process, such as a disease state or a diagnostic or prognostic indicator of a disease or disorder (e.g., an indicator identifying the likelihood of the existence or later development of a disease or disorder). The presence or absence of a biomarker, or the increase or decrease in the concentration of a biomarker, can be associated with and/or be indicative of a particular state or process. Biomarkers can include, but are not limited to, cells or cellular components (e.g., a viral cell, a bacterial cell, a fungal cell, an apoptotic cell, an immune cell, etc.), small molecules, lipids, carbohydrates, nucleic acids, peptides, proteins, enzymes, antigens, and antibodies. A biomarker can be derived from an infectious agent, such as a bacterium, fungus, or virus, or can be an endogenous molecule that is found in greater or lesser abundance in a subject suffering from a disease or disorder as compared to a healthy individual (e.g., an increase or decrease in expression of a gene or gene product).

As used herein, the term “cytotoxic” or “cytotoxicity” refers to killing or damaging cells. In some embodiments of the present disclosure, the cytotoxicity of modified monocytes or macrophages as described herein is improved (e.g., increased cytolytic or phagocytic activity).

As used herein, the term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses (i.e., lentiviral vectors) offer the means to achieve significant levels of gene transfer in vivo.

As used herein, the term “immune effector cell” refers to a macrophage or a monocyte. Monocytes and macrophages are similar types of immune cells that are members of the mononuclear phagocyte system, which is a component of innate immunity.

As used herein, the term “modified” refers to a changed state or structure of a molecule or cell of the present disclosure. Molecules may be modified in many ways, including chemically, structurally, and functionally. Cells may be modified through the introduction of nucleic acids or proteins. For example, in some nonlimiting embodiments, a modified macrophage or monocyte is described that comprises surface-expressed CD47-targeted chimeric antigen receptor (CAR) proteins, and lipid-based particles conjugated to a surface of the modified macrophage or monocyte.

As used herein, the term “immune response” is defined as a cellular response to an antigen that occurs when an immune effector cell identifies antigenic molecules as foreign and induces the formation of antibodies and/or activates immune effector cells to remove the antigen.

As used herein, the term “chimeric antigen receptor” or “CAR” refers to an artificial cell surface receptor that is engineered to be expressed on an immune effector cell and specifically bind to an antigen. CARs may be used as a therapy with adoptive cell transfer. The structure of CAR constructs may be modulated based on the intended target antigen and the specific immune cell type comprising the CAR. Immune effector cells such as monocytes or macrophages may be removed from a patient (e.g., blood, tumor, or ascites fluid) and modified (e.g., using a lentiviral vector expression system) so that they express the CARs specific to a particular form of antigen.

In some embodiments of the present disclosure, the disclosed CAR proteins have a modular design and typically comprise four major components: a single-chain variable fragment (scFv) comprising VL and VH to recognize a target antigen and redirect the specificity of CAR-expressing cells; a hinge domain to provide sufficient flexibility for accessing the antigen; a transmembrane domain to anchor and stabilize the CAR on the cell membrane and provide a functional signaling link between the extracellular and intracellular regions of the CAR; and an intracellular signaling domain to stimulate signal transduction in an immune effector cell. Optimal molecular design of CAR protein constructs can be achieved through many variations of these constituent protein domains. The disclosed CAR proteins may be modified for better activity, stability, expression, production, storage, administration, detection, delivery efficiency, etc. For example, in some embodiments, disclosed CAR proteins may be modified with one or more molecular tags, leader sequences, and/or linker sequences. In various nonlimiting exemplary embodiments, disclosed CD47-targeted CAR proteins may comprise an N-terminal CD8 leader sequence; an anti-CD47 scFv comprising VL and VH sequences connected through a GS linker sequence; CD8 hinge and transmembrane domain sequences; and a C-terminal CD3ζ signaling domain sequence. Disclosed CD47-targeted CAR proteins may be described in U.S. Pat. No. 11,692,034, the entire contents of which is hereby incorporated into the specification.

In various embodiments disclosed herein are modified CAR macrophages (CAR-Ms) for enhanced clearance (efferocytosis) of phagocytosis-resistant, CD47-overexpressing apoptotic cells (ACs). In certain embodiments, therapeutic lipid particles (comprised of β-cyclodextrin (β-CD)) may be tethered on the modified macrophage cell surface for simultaneous drug delivery to alleviate excess lipid burden during CAR-mediated phagocytosis, enhancing CAR-M function and leading to anti-inflammatory effects. The disclosed compositions and methods provide a cell therapy platform for treating various inflammatory diseases where efferocytosis may be impaired.

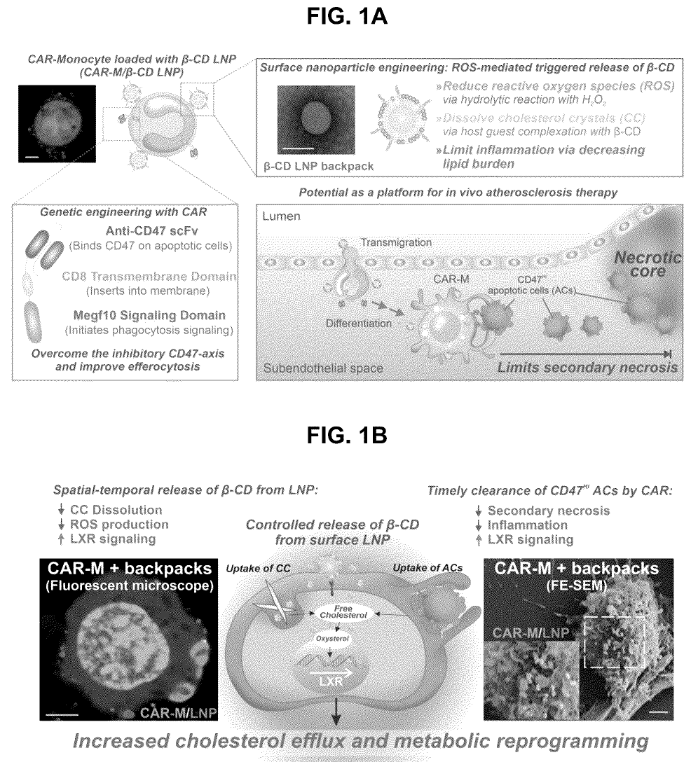

In some nonlimiting exemplary embodiments of the present disclosure, a nanoimmuno-engineering cell platform is described comprised of (i) anti-CD47 CAR-Ms and (ii) surface-anchored HPβ-CD lipid nanoparticles (β-CD LNPs) to target hard-to-clear CD47^{Hi} ACs (FIG. 1A). It was hypothesized that the clearance of CD47^{Hi} ACs could be improved by dual anti-CD47 CAR and β-CD LNP engineering. The propensity of monocytes to localize to inflamed tissues allows them to serve as advanced cell-based-drug delivery vehicles in vivo. It was also hypothesized that CAR-expressing monocytes could mediate phagocytosis against CD47^{Hi} ACs upon differentiation into CAR macrophages in situ at the lesion site in addition to serving as drug delivery vehicles. Combined with the ability of HPβ-CD to solubilize cholesterol crystals (CC) and increase oxysterol metabolism, it is possible to achieve enhanced clearance and reduce atherosclerotic burden via upregulating the LXR signaling pathway in the macrophages (FIG. 1B). To achieve this, genetic engineering and nanotechnology may be combined to manipulate macrophages to break down ACs and CC by increasing lipid catabolism through LXR signaling. This “living factory” ensures that macrophages can efficiently break down lipids without being dilapidated by the immune system. Such metabolic reprogramming leads to increased cholesterol efflux and limits the overall lipid burden.

One embodiment described herein is a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD). In one aspect, the modified macrophage or monocyte is derived from a primary macrophage or monocyte, or wherein the modified macrophage or monocyte is derived from an induced pluripotent stem cell (iPSC). In another aspect, the CD47-targeted CAR proteins comprise an anti-CD47 single-chain variable fragment (scFv) comprising VL and VH; a CD8 hinge domain; a CD8 transmembrane domain; and a CD3ζ signaling domain. In another aspect, the CD47-targeted CAR proteins comprise an amino acid sequence having at least 90-99% identity to SEQ ID NO: 1. In another aspect, the CD47-targeted CAR proteins comprise an amino acid sequence of SEQ ID NO: 1. In another aspect, the lipid-based particles are lipid nanoparticles (LNPs) or liposomes. In another aspect, the lipid-based particles are LNPs comprising one or more of 1,2-dimyristoyl-sn-glycero-3-phosphocholine (DMPC), 1,2-dioleoyl-sn-glycero-3-phosphate (DOPA), cholesterol, or 1,2-distearoyl-sn-glycero-3-phosphoethanolamine-N-[amino(polyethylene glycol)-2000](DSPE-PEG(2000)). In another aspect, the β-CD is hydroxypropyl β-CD (HPβ-CD). In another aspect, the β-CD is modified with phenylboronic acid pinacol ester (PBAP). In another aspect, the lipid-based particles comprise a surface-conjugated anti-CD45 antibody that binds to CD45 expressed on the surface of the modified macrophage or monocyte. In another aspect, the lipid-based particles have a mean diameter of about 100 nm to about 350 nm. In another aspect, the lipid-based particles have a negative zeta potential of about −35 mV to about −50 mV. In another aspect, about 50 lipid-based particles to about 300 lipid-based particles are conjugated to the surface of the modified macrophage or monocyte. In another aspect, about 100 lipid-based particles to about 200 lipid-based particles are conjugated to the surface of the modified macrophage or monocyte. In another aspect, the modified macrophage or monocyte has enhanced phagocytosis and transmigration properties.

Another embodiment described herein is a pharmaceutical composition comprising any of the modified macrophages or monocytes described herein.

Another embodiment described herein is a method of treating atherosclerosis in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD). In one aspect, the β-CD is released from the lipid-based particles in response to elevated levels of reactive oxygen species (ROS) in the subject. In another aspect, the modified macrophage or monocyte reduces an amount of CD47-overexpressing apoptotic cells in the subject. In another aspect, the CD47-overexpressing apoptotic cells are apoptotic foam cells. In another aspect, the modified macrophage or monocyte reduces an amount of insoluble cholesterol in the subject. In another aspect, the modified macrophage or monocyte reduces inflammation in the subject.

Another embodiment described herein is a method of treating cardiovascular disease in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

Another embodiment described herein is a method of treating inflammation in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

Another embodiment described herein is a method of treating a chronic inflammatory disease in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

Another embodiment described herein is a method of treating a wound in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

Another embodiment described herein is a method of treating a spinal cord injury in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A modified macrophage or monocyte comprising:
   surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).
Clause 2. The modified macrophage or monocyte of clause 1, wherein the modified macrophage or monocyte is derived from a primary macrophage or monocyte, or wherein the modified macrophage or monocyte is derived from an induced pluripotent stem cell (iPSC).
Clause 3. The modified macrophage or monocyte of clause 1 or 2, wherein the CD47-targeted CAR proteins comprise an anti-CD47 single-chain variable fragment (scFv) comprising VL and VH; a CD8 hinge domain; a CD8 transmembrane domain; and a CD3ζ signaling domain.
Clause 4. The modified macrophage or monocyte of any one of clauses 1-3, wherein the CD47-targeted CAR proteins comprise an amino acid sequence having at least 90-99% identity to SEQ ID NO: 1.
Clause 5. The modified macrophage or monocyte of any one of clauses 1-4, wherein the CD47-targeted CAR proteins comprise an amino acid sequence of SEQ ID NO: 1.
Clause 6. The modified macrophage or monocyte of any one of clauses 1-5, wherein the lipid-based particles are lipid nanoparticles (LNPs) or liposomes.
Clause 7. The modified macrophage or monocyte of any one of clauses 1-6, wherein the lipid-based particles are LNPs comprising one or more of 1,2-dimyristoyl-sn-glycero-3-phosphocholine (DMPC), 1,2-dioleoyl-sn-glycero-3-phosphate (DOPA), cholesterol, or 1,2-distearoyl-sn-glycero-3-phosphoethanolamine-N-[amino(polyethylene glycol)-2000](DSPE-PEG(2000)).
Clause 8. The modified macrophage or monocyte of any one of clauses 1-7, wherein the β-CD is hydroxypropyl β-CD (HPβ-CD).
Clause 9. The modified macrophage or monocyte of any one of clauses 1-8, wherein the β-CD is modified with phenylboronic acid pinacol ester (PBAP).
Clause 10. The modified macrophage or monocyte of any one of clauses 1-9, wherein the lipid-based particles comprise a surface-conjugated anti-CD45 antibody that binds to CD45 expressed on the surface of the modified macrophage or monocyte.
Clause 11. The modified macrophage or monocyte of any one of clauses 1-10, wherein the lipid-based particles have a mean diameter of about 100 nm to about 350 nm.
Clause 12. The modified macrophage or monocyte of any one of clauses 1-11, wherein the lipid-based particles have a negative zeta potential of about −35 mV to about −50 mV.
Clause 13. The modified macrophage or monocyte of any one of clauses 1-12, wherein about 50 lipid-based particles to about 300 lipid-based particles are conjugated to the surface of the modified macrophage or monocyte.
Clause 14. The modified macrophage or monocyte of any one of clauses 1-13, wherein about 100 lipid-based particles to about 200 lipid-based particles are conjugated to the surface of the modified macrophage or monocyte.
Clause 15. The modified macrophage or monocyte of any one of clauses 1-14, wherein the modified macrophage or monocyte has enhanced phagocytosis and transmigration properties.
Clause 16. A pharmaceutical composition comprising the modified macrophage or monocyte of any one of clauses 1-15.
Clause 17. A method of treating atherosclerosis in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).
Clause 18. The method of clause 17, wherein the β-CD is released from the lipid-based particles in response to elevated levels of reactive oxygen species (ROS) in the subject.
Clause 19. The method of clause 17 or 18, wherein the modified macrophage or monocyte reduces an amount of CD47-overexpressing apoptotic cells in the subject.
Clause 20. The method of any one of clauses 17-19, wherein the CD47-overexpressing apoptotic cells are apoptotic foam cells.
Clause 21. The method of any one of clauses 17-20, wherein the modified macrophage or monocyte reduces an amount of insoluble cholesterol in the subject.
Clause 22. The method of any one of clauses 17-21, wherein the modified macrophage or monocyte reduces inflammation in the subject.
Clause 23. A method of treating cardiovascular disease in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).
Clause 24. A method of treating inflammation in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).
Clause 25. A method of treating a chronic inflammatory disease in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).
Clause 26. A method of treating a wound in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).
Clause 27. A method of treating a spinal cord injury in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

### EXAMPLES

### Example 1

### Materials and Methods

4-(Hydroxymethyl)phenylboronic acid pinacol ester was purchased from Sigma-Aldrich (St. Louis, MO). Hydroxypropyl-β-cyclodextrin (HPβ-CD) was purchased from Fisher Scientific (Waltham, MA). 1,1′-Carbonyldiimidazole was purchased from Sigma-Aldrich (St. Louis, MO). Methylene Chloride was purchased from Fisher Scientific (Waltham, MA). Dimethyl sulfoxide was purchased from Sigma-Aldrich (St. Louis, MO). 4-dimethylaminopyridine was purchased from VWR (Radnor, PA). 1,2-dimyristoyl-sn-glycero-3-phosphocholine (DMPC), 1,2-dioleoyl-sn-glycero-3-phosphate (DOPA), 1,2-distearoyl-sn-glycero-3-phosphoethanolamine-N-[amino(polyethylene glycol)-2000](DSPE-PEG(2000))-maleimide (ammonium salt), TopFluor cholesterol were purchased from Avanti Polar Lipids (Alabaster, AL). Chloroform was purchased from Fisher Scientific (Waltham, MA). Hydrogen peroxide was purchased from Sigma-Aldrich (St. Louis, MO). DCFDA was purchased from Sigma-Aldrich (St. Louis, MO). Presto Blue Cell Viability Reagent was purchased from Fisher Scientific (Waltham, MA). Nile Red was purchased from Sigma-Aldrich (St. Louis, MO). Sodium sulfate was purchased from Sigma-Aldrich (St. Louis, MO). Sodium chloride was purchased from Sigma-Aldrich (St. Louis, MO). Chloroform-d₁ was purchased from VWR (Radnor, PA). Potassium bromide (IR grade powder) was purchased from VWR (Radnor, PA). Cholesterol was purchased from Sigma-Aldrich (St. Louis, MO). Trypan blue 0.1% solution was purchased from VWR (Radnor, PA). BSA was purchased from Biosciences (Warrington, PA). Rutin hydrate was purchased from Sigma-Aldrich (St. Louis, MO). Protein Assay Dye Reagent Concentrate was purchased from Biorad (Hercules, CA). Sylgard™ 184 Silicone Elastomer was purchased from Electron Microscopy Sciences (Hatfield, PA).

### Synthesis of PBAP-CD

Phenylboronic acid pinacol ester (PBAP) was conjugated to HPβ-CD using a previously established method. Briefly, PBAP (550 mg) and CDI (760 mg) were added to 7 mL of anhydrous DCM. The reaction flask was flushed with argon gas and stirred for 30 min at RT. The reaction mixture was washed twice with deionized water and once with brine. The organic solvent was dried using sodium sulfate and then vacuumed for a duration of 24 h. The PBAP-activated CDI (430 mg) was added to 9 mL of DMSO along with DMAP (270 mg) and HPβ-CD (106 mg). The reaction vessel was flushed with argon gas and stirred for 48 h at RT. The mixture was then precipitated in deionized water and collected by centrifugation. The pellet was dried under a vacuum for 24 h.

### ¹H-NMR and FTIR Characterization of PBAP-CD

The PBAP-CD was characterized by Proton Nuclear Magnetic Resonance (¹H-NMR) and Infrared Spectroscopy (IR). For ¹H-NMR analysis, 1 mg of PBAP-CD was dissolved in deuterated chloroform, and the sample was run on a BrukerAVANCE Neo 500 MHz NMR. For IR analysis, 1 mg of PBAP-CD was added to 100 mg of dried KBr powder. The powder was ground to a fine powder using a mortar and pestle. Approximately 50 mg of this was then used to create a KBr disc using a minipress. The spectrum was then captured using a Thermo Fisher Nicolet iS10.

### Preparation and Characterization of β-CD LNP

1,2-dimyristoyl-sn-glycero-3-phosphocholine (DMPC) and 1,2-dioleoyl-sn-glycero-3-phosphate (sodium salt) (DOPA) were constituted in 20 mM stock solutions in chloroform. The stock solutions were stored in −20° C. until use. The chloroform was vacuumed off and the lipids were redispersed in ethanol. DSPE-PEG(2000)-maleimide (ammonium salt) was reconstituted as 1 mg/mL. DMPC (81 μL), DOPA (51 μL), and DSPE-PEG(2000)-maleimide (1 μL) were added to make a 4% ethanolic solution in HEPES pH 7.4 buffer. The solution was heated at 65° C. for 1 h. PBAP-CD (5 mg) dissolved in methanol was added drop-wise to the lipid solution, and vortexed for 3.75 min. The methanol was removed by evaporation for 2 h at 65° C. The formed β-CD LNPs were then stored at 4° C. until use. Hydrodynamic diameter, zeta potential, and particle concentration of the β-CD LNPs were measured using a Malvern ZetaSizer Nano Series and NanoSight NS 3000 (Westborough, MA). For XPS analysis, 10 μL of the β-CD LNP solution was dried on a cleaned silicon substrate and analyzed using a Thermo Scientific™ K-Alpha™ X-ray Photoelectron Spectrometer (XPS) System with a monochromated X-ray source (Al-Kα) and a base pressure of <5×10⁻⁸ mbar. TEM images of the β-CD LNPs were acquired using a JEOL transmission electron microscope (JEM1010; JEOL, Japan) with an accelerating voltage of 80 kV.

β-CD Release Kinetics from LNPs

The amount of β-CD released from the LNPs was determined by complexing the free 3-CD to rutin. 4 μL of rutin (1 mM in methanol) was added to 2 mL β-CD LNPs (1 mg/mL in HEPES pH 7.4 buffer). The sample was aliquoted into 300 μL portions, followed by adding H₂O₂ (10 mM stock). The sample was then pipetted into a 96 well plate at 100 μL per well. The fluorescence was measured at various time points using a microplate reader. The calibration curve was generated by adding 100 μL rutin hydrate at various concentrations (0, 2, 4, 6, 8 mM) along with β-CD. The calibration curve was measured and determined individually for each time point. The same protocol was repeated with some minor changes to facilitate total hydrolysis. The β-CD LNPs were aliquoted, followed by the addition of 1 mM H₂O₂. The sample was incubated at RT overnight. Rutin was then added, incubated for 30 min, and fluorescence was measured.

### Cell Culture

Cells of the human monocytic cell line THP-1 were cultured in complete RPMI-1640 medium (Gibco, Thermo Fisher Scientific) with 10% FBS, 1% penicillin-streptomycin and 0.1% β-mercaptoethanol. The THP-1 cells were differentiated into macrophage-like cells with 50 ng/mL phorbol 12-myristate 13-acetate (PMA) (Tocris Bioscience) for 48 h. For polarization into M1-like macrophages, the cells were incubated with 100 ng mL-1 LPS and 50 ng/mL IFN-γ for 24 h after differentiation. HEK293 and MCF-7 cells were cultured in Dulbecco's modified Eagle's medium (DMEM) supplemented with 10% FBS, 1% penicillin-streptomycin, 1% non-essential amino acids (NEAA) and L-glutamine. HUVECs were cultured in endothelial growth medium (DMEM/F12, 2% FBS, 1 μg/mL hydrocortisone, 5 ng/mL EGF, 10 ng/mL FGF-2, 20 μg/mL heparin sulphate, 250 ng/mL insulin, and 100 U/mL penicillin). The cells were cultured at 37° C. in a humidified atmosphere containing 5% CO₂.

### ROS Scavenging Assay

UV-induced photodegradation of rhodamine B was used to examine the ROS-scavenging effect of β-CD. Briefly, 1.68 mM of H₂O₂ was subjected to UV-irradiation for 1 h and then mixed with rhodamine B, with a final rhodamine B concentration of 10 μM. The mixture (with and without LNPs) was further irradiated for 15-20 min and the absorbance of rhodamine B was recorded at 553 nm using a Varian Cary50 spectrophotometer. For intracellular ROS measurement, THP-1 cells were seeded at 30,000 cells per well and differentiated for 48 h. Thereafter, the media was replaced with serum-free media and the cells were incubated with 50 μM H₂O₂ in addition to varying doses of β-CD LNPs for 1 h at 37° C. The cells were then washed twice with PBS and incubated with 2′-7′-Dichlorodihydrofluorescein diacetate DCFDA (100 μM) for 2 h at 37° C. Fluorescence was then measured (excitation 485 nm, emission 535 nm). The amount of ROS scavenged was determined by plotting a calibration curve with the negative and positive control.

### Cell Viability

Cell viability was measured by incubating macrophages with β-CD LNPs at varying concentrations for 24 h. The Presto Blue assay (10% volume ratio to cell medium; Thermo Fisher Scientific) was then carried out according to the manufacturer's instructions. A calibration plot was also plotted using positive and negative control. The concentration of β-CD LNPs refers to the concentration of β-CD assuming 100% encapsulation.

### Cholesterol Crystal Preparation and Dissolution

Cholesterol monohydrate crystals were prepared as reported previously. Briefly, cholesterol was dissolved in hot acetone at 20 mg/mL. The opaque solution was heated to 90° C. to dissolve, followed by precipitation in an ice bath. This cycle was repeated 6 times. In the last cycle, nuclease-free water was added to 10% of the final volume to create monohydrate cholesterol crystals (CC). The CC were collected by centrifugation, and the acetone/water mixture was decanted. The dried crystals were further reduced in size using a probe ultrasonicator (20 kHz, 5 Watts) in sterile, tissue grade PBS for 1 h on ice. For fluorescently labeled CC, a small amount of BODIPY-labeled cholesterol (TopFluor-cholesterol®, Avanti, Alabaster, AL) was doped into the cholesterol solution in acetone. The capacity of β-CD LNPs to dissolve CC was evaluated by incubating different concentrations of LNPs with CC at a fixed H₂O₂ concentration for 16 h at 37° C. The samples were subsequently filtered with a 0.22 μm filter and supernatant (filtrate) was collected for fluorescence analysis using a Tecan microplate reader (Mannedorf, Switzerland) and an excitation wavelength of 488 nm and emission wavelength of 550 nm. The crystalline fraction was obtained by incubating the filters with hot methanol and the fluorescence was recorded.

### Intracellular CC Content

Macrophages were incubated with 2 μg of CC for 3 h, followed by treatment with β-CD LNPs for 24 h. The cells were washed three times with PBS, once with 0.1% Trypan Blue solution, followed by three times with PBS. The absorbance of the BODIPY-cholesterol was then measured.

### Foamy Macrophage Staining

To evaluate the capability of β-CD LNP-backpacked macrophages to resist lipid accumulation, Nile Red staining was employed. In short, LNP-backpacked THP-1 cells were seeded at 30,000 cells per well in a 96-well plate and differentiated with PMA. After differentiation, the cells were incubated with PBS or ox-LDL (25 μg/mL) for 24 h in serum-free media, followed by the addition of an equal volume of media with FBS for another 24 h. The cells were then washed, fixed, and stained with 5 μL of 1 mg/mL Nile Red for 30 min at RT. The cells were subsequently washed twice, and fluorescence was recorded using a Tecan microplate reader (excitation 488, emission 550).

Antibody Coupling to β-CD LNPs Anti-CD45 antibodies were conjugated to the LNPs using thiol-maleimide coupling. For a typical reaction, 10 μg of anti-human CD45 antibody (BioLegend, San Diego, CA) reconstituted in a small volume of PBS containing 2 mM EDTA at pH 7.4 was activated using Traut's reagent for 1 h at RT. Subsequently, 900 μL of LNPs were added, and the solution was left on a rotatory shaker for 16 h at 4° C. Following the reaction, 1 μM L-cysteine was added to quench any unreacted maleimide groups. Unreacted mAbs were purified with Vivaspin 500 (Sartorius, Gotten, Germany). The antibody-coupled LNPs were stored at 4° C. until use. The concentration of the antibody on the surface of the LNPs was determined by the Bradford assay and the absorbance measured at 595 nm. A calibration curve was determined using BSA (Polysciences, Warrington, PA).

### Backpacking of β-CD LNPs to THP-1 Cells

The β-CD LNP backpacks were loaded onto the cells using a modified procedure as described previously. For a typical experiment, 1.0×10⁶ cells were incubated with 90 μL of the LNPs in Hank's Balanced Salt Solution (HBSS) (Gibco, Thermo Fisher Scientific) for 1 h at 37° C. Thereafter, the cells were washed three times with HBSS and used for subsequent experiments. The loading efficiency at different concentrations was calculated by the following formula:

$$Loading Efficiency ( L E \% ) = ( 1 - {C}_{f} / {C}_{t} ) \times 100 \%$$

where Cr is the fluorescence of the amount of free, non-loaded LNPs in the supernatant and C, is the fluorescence of the total amount of LNPs. To determine dose-dependent loading, various concentrations of LNPs were incubated with 1.0×10⁶ cells, washed three times, and analyzed by flow cytometry using a Beckman Coulter Gallios Cytometer (Beckman Coulter, Brea, CA), equipped with 405 nm, 488 nm, and 638 nm lasers. Data were analyzed using Beckman Coulter Kaluza version 1.2 software. To visualize the LNPs on the THP-1 cells, the backpacked cells in suspension were labeled with CMTPX (C₄₂Hᵣ₀ClN₃O₄; Invitrogen), washed, and adhered onto the glass coverslips using a previously described method. The macrophages were fixed and imaged using a Zeiss LSM800 confocal microscope.

### Surface Imaging and Quantification of β-CD LNPs

Characterization of CAR-Ms backpacked with β-CD LNPs was conducted using field emission scanning electron microscopy (FE-SEM). To prepare the sample, 5×10⁴ cells were differentiated on a 10 mm×10 mm size of glass substrate and cultured for 2 days. The cells were washed three times with DPBS and fixed using 4% formaldehyde in DPBS for 10 min at RT. The fixed cells were subjected to graded dehydration using various concentrations of ethanol (30, 50, 70, 90, and 100%, each for 10 min) and hexamethyldisilazane (HMDS) (50 and 100%, each for 10 min). The dehydrated cells were air-dried overnight on a clean bench. Afterwards, the glass with the dried cells was mounted onto aluminum stubs and coated with 20 nm of gold using a sputter coater. The morphology of the CAR-Ms with β-CD LNPs was then observed using FE-SEM (10 kV, Carl Zeiss, Germany). The images obtained from FE-SEM were analyzed for morphological changes due to the β-CD LNPs. CAR-Ms and β-CD LNP backpacks in the FE-SEM image were pseudocolored using Adobe photoshop. To quantify surface-coupled β-CD LNPs, DSPE-PEG-biotin was incorporated into the LNPs via post-insertion for 15 min at 55° C. The purified antibody-coupled LNPs were prepared and conjugated to the cell surface as described in the above section. Surface detection of LNPs was achieved by staining the cells with streptavidin-AlexaFluor 647 and detection by flow cytometry.

### Lentiviral Vector Design and Production of CD47-Targeted CAR Proteins

The humanized anti-CD47 scFv was designed based on previous work for engineering anti-CD47 CAR T-cells. The lentiviral expression vector was custom cloned by VectorBuilder. For lentivirus production, HEK293T cells were cultured to 60-70% confluency before transfecting with the expression vector, the psPAX2 packaging vector (Addgene #12260), and the envelope vector pMD2.G (Addgene #12259) at a ratio of 4:3:1, respectively, using Lipofectamine 3000. The supernatant containing lentiviral particles was harvested 48-72 h post-transfection, filtered with a 0.45 μm syringe filter, and the virus was isolated using the PEG6000 precipitation method. Lentivirus titer was determined using the qPCR Lentivirus Titer Kit (Applied Biological Materials, Canada) according to the manufacturer's instructions. The amino acid sequences of the CD47-targeted CAR protein are provided in Table 1.

TABLE 1
Amino Acid Sequences of CD47-Targeted CAR Protein

| Full Amino Acid Sequence | SEQ ID NO |
|---|---|
| MALPVTALLLPLALLLHAARPEIVLTQSPATLSLSPGERATLSCRASQSISDYLHW | 1 |
| YQQKPGQAPRLLIYFASQRATGIPARFSGSGSGTDFTLTISSLEPEDFAVYYCQQG | (443 AA) |
| HGFPRTFGGGTKVEIKGGGGSGGGGSGGGGSEVQLVESGGGLVQPGGSLRLSCAAS | |
| GFTFSGYGMSWVRQAPGKGLEWVATITSGGTYTYYPDSVKGRFTISRDNAKNSLYL | |
| QMNSLRAEDTAVYYCARSLAGNAMDYWGQGTLVTVSSTTTPAPRPPTPAPTIASQP | |
| LSLRPEACRPAAGGAVHTRGLDFACDIYIWAPLAGTCGVLLLSLVITLYCRVKFSR | |
| SADAPAYQQGQNQLYNELNLGRREEYDVLDKRRGRDPEMGGKPQRRKNPQEGLYNE | |
| LQKDKMAEAYSEIGMKGERRRGKGHDGLYQGLSTATKDTYDALHMQALPPR | |
| Sequence Element Regions/Amino Acids | Sequence Element |
| MALPVTALLLPLALLLHAARP | CD8 leader sequence |
| | (AA 1-20) |
| EIVLTQSPATLSLSPGERATLSCRASQSISDYLHWYQQKPGQAPRLLIYFASQRAT | scFv VL sequence |
| GIPARFSGSGSGTDFTLTISSLEPEDFAVYYCQQGHGFPRTFGGGTKVEIK | (AA 21-128) |
| GGGGSGGGGSGGGGS | GS linker sequence |
| | (AA 129-143) |
| EVQLVESGGGLVQPGGSLRLSCAASGFTFSGYGMSWVRQAPGKGLEWVATITSGGT | scFv VH sequence |
| YTYYPDSVKGRFTISRDNAKNSLYLQMNSLRAEDTAVYYCARSLAGNAMDYWGQGT | (AA 144-261) |
| LVTVSS | |
| TTTPAPRPPTPAPTIASQPLSLRPEACRPAAGGAVHTRGLDFACDIYIWAPLAGTC | CD8 |
| GVLLLSLVITLYC | hinge/transmembrane |
| | domain sequence |
| | (AA 262-330) |
| RVKFSRSADAPAYQQGQNQLYNELNLGRREEYDVLDKRRGRDPEMGGKPQRRKNPQ | CD3ζ sequence |
| EGLYNELQKDKMAEAYSEIGMKGERRRGKGHDGLYQGLSTATKDTYDALHMQALPP | (AA 331-443) |
| R | |

### Transduction and Selection of THP-1 Cells by FACS

THP-1 cells (1.0×10⁵ cells) were transduced with lentivirus at various multiplicity of infection (MOI), ranging from 0.5 to 20. Successfully transduced cells were expanded and sorted by FACS using the MoFlo Astrios cell sorter (Beckman Coulter, Brea, CA).

### Quantitative Reverse Transcription Polymerase Chain Reaction (RT-qPCR)

Depending on the experiments, the total RNA was isolated from cells either 9 h- or 24 h-post incubation for CC-related studies and efferocytosis experiments, respectively, using TRIzol Reagent (Life Technologies, MA). The total RNA was reverse transcribed to cDNA using SuperScriptIII First-Strand Synthesis System (Life Technologies, MA). The qPCR reactions were performed using a StepOnePlus RT-PCR system (Applied Biosystems) with Power SYBR Green PCR master mix (Applied Biosystems). The gene expression levels were reported in fold change values relative to control and normalized against glyceraldehyde-3-phosphate dehydrogenase (GAPDH) gene expression. The primers used are provided in Table 2.

TABLE 2
RT-qPCR Primer Sequences

| | | SEQ ID | | SEQ ID |
|---|---|---|---|---|
| Gene | Forward Primer | NO | Reverse Primer | NO |
| GAPDH | CATGTTCCAATATGATTCCACC | 2 | GATGGGATTTCCATTGATGAC | 3 |
| IL-1β | ATGATGGCTTATTACAGTGGCAA | 4 | GTCGGAGATTCGTAGCTGGA | 5 |
| IL-6 | AAACAACCTGAACCTTCCAAAGA | 6 | GCAAGTCTCCTCATTGAATCCA | 7 |
| ABCA1 | ACATCCTGAAGCCAATCCTGA | 8 | CTCCTGTCGCATGTCACTCC | 9 |
| ABCG1 | ATTCAGGGACCTTTCCTATTCGG | 10 | CTCACCACTATTGAACTTCCCG | 11 |
| CYP27A1 | CGGCAACGGAGCTTAGAGG | 12 | GGCATAGCCTTGAACGAACAG | 13 |
| IL-10 | GCTCCTGAGGTATGGAATAGAGTCC | 14 | TATGTGTCATTTGCGGGGGC | 15 |
| MERTK | CTCTGGCGTAGAGCTATCACT | 16 | AGGCTGGGTTGGTGAAAACA | 17 |
| CD206 | CTACAAGGGATCGGGTTTATGGA | 18 | TTGGCATTGCCTAGTAGCGTA | 19 |
| TFEB | ACCTGTCCGAGACCTATGGG | 20 | CGTCCAGACGCATAATGTTGTC | 21 |
| CCL2 | CAGCCAGATGCAATCAATGCC | 22 | CAGCCAGATGCAATCAATGCC | 23 |
| NLRP3 | CCACAAGATCGTGAGAAAACCC | 24 | CGGTCCTATGTGCTCGTCA | 25 |
| TNF-α | CTGCTGCACTTTGGAGTGAT | 26 | AGATGATCTGACTGCCTGGG | 27 |

### Detection of Surface CAR by Protein L

The detection and quantification of CAR on THP-1 cells were performed using Protein L. Briefly, 1.0×10⁶ cells were washed three times with 1×PBS and resuspended in 0.5 mL of FACS buffer (1×PBS, 25 mM HEPES, 1% FBS). The cells were stained with 1 μg of biotinylated protein L (GenScript, Piscataway, NJ) for 45 min at 4° C. The cells were subsequently washed three times with ice-cold buffer, then stained with AlexaFluor 647-streptavidin for 30 min at 4° C. The cells were analyzed by flow cytometry as described above.

### CD47 Bead Preparation and Phagocytosis Assay

Liposome-coated beads containing CD47 mimicking CD47-rich apoptotic bodies were prepared using a previously described method with some modifications. Small unilamellar vesicles (SUVs) containing 95% DMPC, 2% Ni²⁺-DGS-NTA, 0.5% PEG5000-PE and 0.5% Liss Rhod-PE were prepared using the thin film hydration method, sonicated, and cleared by 33 freeze thaw cycles. Silica microbeads (3 μm in size; Bangs Labs, IN) were mixed with the SUVs at a 2 mM final concentration for 30 min at RT on a rotatory shaker in the dark. The liposome-coated beads were washed twice with PBS, resuspended in PBS+0.1% w/v BSA, and incubated with recombinant human CD47-his (Sino Biological) for an additional 45 min. The CD47-coupled beads were further washed twice with PBS and resuspended in PBS+0.1% w/v BSA. For phagocytosis assays, control macrophages and CAR-M were incubated with the beads for 2 h at 37° C., washed three times with PBS, and fixed with 4% formaldehyde and stained with anti-CD11b antibody (control) or anti-GFP antibody (CAR-M) prior to imaging with a Zeiss LSM800 confocal microscope. The internalized beads were quantified using ImageJ.

### Generating CD47^{Hi} ACs and Standard ACs

MCF-7 cells (1.0×10⁵ cells) were seeded into a 24-well plate and stimulated with TNF-α at 50 ng/mL for 48 h. The cells were then treated with 1 μM of staurosporine (STS) for 4 h. The expression of CD47 was confirmed by flow cytometry and fluorescence microscopy. For standard AC preparation, Jurkat cells (2.0×10⁶ cells) in PBS were seeded into a 6 cm dish and irradiated under a UV lamp for 5 min. The cells were redispersed in growth media and cultured for another 2-4 h prior to further experimentation.

### Phagocytosis of ACs and Quantification by Cell Tagging

Control (untransduced) THP-1 cells or CAR-expressing THP-1 cells were seeded at 1.0×10⁵ cells per well in a 48-well plate and differentiated with 50 ng/mL PMA for 48 h. CMTPX-labeled CD47^{Hi} MCF-7 ACs were fed to the phagocytes at a ratio of 3 AC:1 macrophage for 2 h at 37° C. Subsequently, the media were gently aspirated off and the wells washed with warm PBS three times to remove any unbound ACs. The macrophages were fixed and stained with anti-CD11b antibody (control) or anti-GFP antibody (CAR-M). The macrophages (at least 50 phagocytes per field) in 4-5 field of views were imaged using either a ZOE™ Fluorescent Cell Imager or a Zeiss LSM800 confocal microscope. For time-lapse imaging, a Nikon Eclipse Ti-E microscope was used to image the macrophages incubated with ACs for a total duration of 1 h. To further distinguish the proportion of cells that were partially engulfed or completely engulfed, the proportion of fluorescent-positive cells were calculated using an automated method run with python slightly modified from the CellTagging Script previously developed. Briefly, the background image was translated into binary images by thresholding the green-fluorescent signal. The Otsu method was utilized to determine the threshold for detecting green fluorescence. The watershed method was used to segment the binary image into individual cells. The detected cells were then filtered to remove cell aggregates and debris. The intensity of the red fluorescent signal of interest was then quantified to determine the colocalization of green and red fluorescent signal, as well as the percentage of cells with fluorescence to cells with minimal fluorescent signals to cells with no red fluorescence based on set thresholds in the processed image. The processes were run with the Python 3.11.1 and its libraries: scikit-image 0.19.2, numpy 1.24.0, matplotlib 3.6.2, seaborn 0.12.2, jupyter 5.2.

### Activation of HUVECs

Stimulation of HUVECs was achieved by treating the cells with TNF-α at a concentration ranging from 0-10 ng/mL for at least 24 h at 37° C. The cells in 4-5 view fields were imaged using a ZOE™ Fluorescent Cell Imager and fluorescence analyzed by ImageJ.

### Cytotoxicity of CAR-Ms

The day prior to the experiment, 3.0×10⁵ HUVECs were seeded into a 6 well plate. An equal volume of 3.0×10⁵ control macrophages or CAR-Ms in complete RPMI media (800 μL) was added to the H UVECs. To test the toxicity of CD47-blockade, H UVECs were pre-treated with 10 μg/mL of CD47 antibodies for 30 min prior to the addition of macrophages. The cells were co-cultured for 24 h at 37° C. and analyzed by flow cytometry. Toxicity was measured by calculating the percentage of cells positive for 4′,6-diamidino-2-phenylindole (DAPI) and CD31.

### Fabrication of the Microfluidic Device

The microfluidic device was fabricated using Poly(dimethylsiloxane) (PDMS, Slygard 184, Dow Corning, USA) and mold printed with polycaprolactone (PCL) filaments. The microfluidic model was designed in Sketchup (Google, Inc., Mountain View, CA), exported as STL files, and translated into a printable g-code file using Perfactory Rapid Prototype (RP) (EnvisionTEC, Inc., Dearborn, MI). The g-code was exported to the 3D Bioplotter where it was assigned the material file for polycaprolactone (PCL) (Lot MKCL2159, avg Mn 45k). PCL was loaded into the high-temperature cartridge fitted with a 24 G, 4 mm in length, stainless steel luer lock needle. PCL was printed at a temperature of 125° C. and 6 bar pressure at a speed of 1 mm/s at 37° C. build plate on a plastic 10 cm petri dish. PDMS secondary templates were prepared using this PCL master by pouring a PDMS prepolymer-catalyst mixture onto the 3D printed structure and cured at a low temperature (37° C.) for 24 h to prevent the melting of PCL. The PDMS cast was then attached to a clean glass slide by plasma treatment at 70 W for 2.05 min (CUTE, Femto Science Inc., Korea). The polar silanol groups on the plasma-treated surfaces of both materials formed irreversible bonds, allowing the assembled device to withstand high pressure.

### Macrophage Penetration/Migration in Different GeIMA Concentrations

A set of 5%, 6.5%, 7.5%, and 10% GeIMA solutions (w/v) were prepared in complete macrophage media in the presence of 1×10⁶ apoptotic cells/mL and 0.5% Irgacure (w/v). GeIMA hydrogels were obtained after cross-linking under UV (312 nm, at a 3 cm distance), washed with PBS, and incubated overnight with 1.0×10⁶ CAR-Ms/mL or 2.0×10⁶ CAR-Ms loaded with LNPs/mL. Hydrogels prepared without ACs were used as control. The next day, hydrogels were washed with PBS twice, and transferred into a new 96 well plate containing PBS to observe under fluorescence microscope.

### Microfluidic Culturing of HUVECs and Transmigration of THP-1 Macrophages

The GeIMA layer of the model was formed in the microfluidic device by injecting uncross-linked 6.5% (w/v) GeIMA with 0.5% (w/v) Irgacure, 0.75 μg/mL of CCl2, and 2.0×10⁶ ACs/mL to the shallower channel. Then, the GeIMA was cross-linked under UV treatment for 1 min. After cross-linking, Tris-HCL buffer containing 0.5 mg/mL of dopamine hydrochloride (Sigma-Aldrich, USA) was injected into the deeper channel to incubate cross-linked GeIMA for 45 min. The dopamine coating promoted the attachment of HUVECs on the surfaces of GelMA. Subsequently, PBS was used to wash the residual dopamine from the non-GelMA surfaces. HUVECs were then seeded in the deeper channel (2.0×10⁶ cells/mL). Seeded HUVECs were incubated at 37° C. until the complete coverage of the GelMA layer with daily media change. The layer of HUVECs was activated by adding media with 1 ng/mL of TNF-α. 24 h after the activation, macrophages stained with CMTPX were injected into the shallower channel and incubated overnight. The next day, unattached macrophages were washed away twice with PBS. The extent of transmigration of the macrophages into the GeIMA layer was studied using a Zeiss LSM710 (Oberkochen, Germany) confocal laser scanning electron microscope and the images were analyzed using NIH Image J.

### Statistical Analysis

All statistical analysis was performed in Prism 9.5.0 (GraphPad, Inc.). The specific statistical test used is indicated in each figure description. Error bars denote 95% confidence intervals of the mean.

### Example 2

### Synthesis and Characterization of β-CD Lipid Nanoparticles (β-CD LNPs)

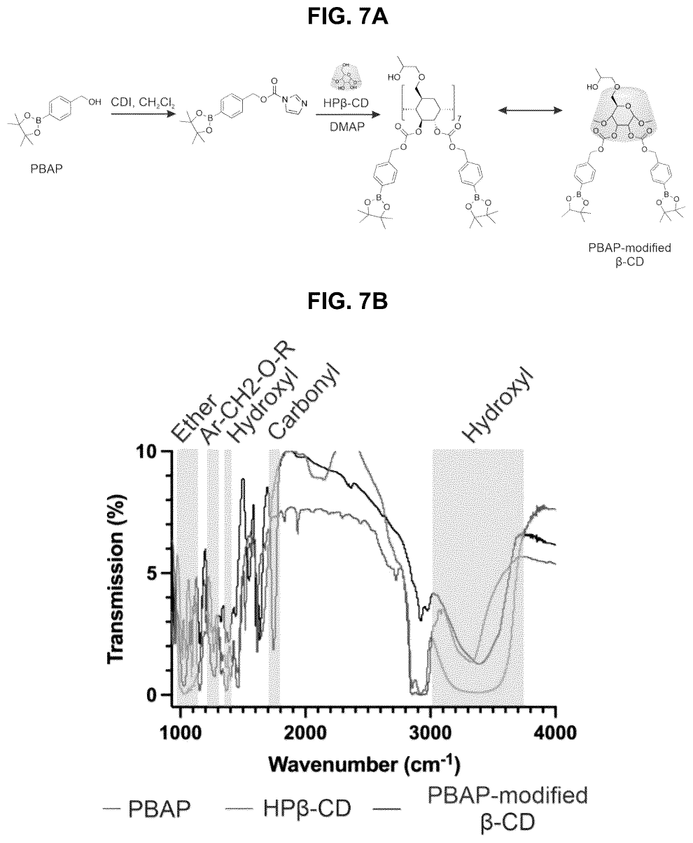

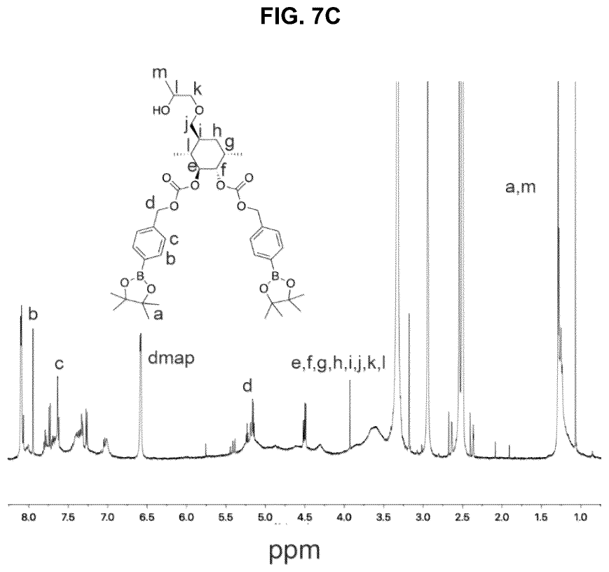

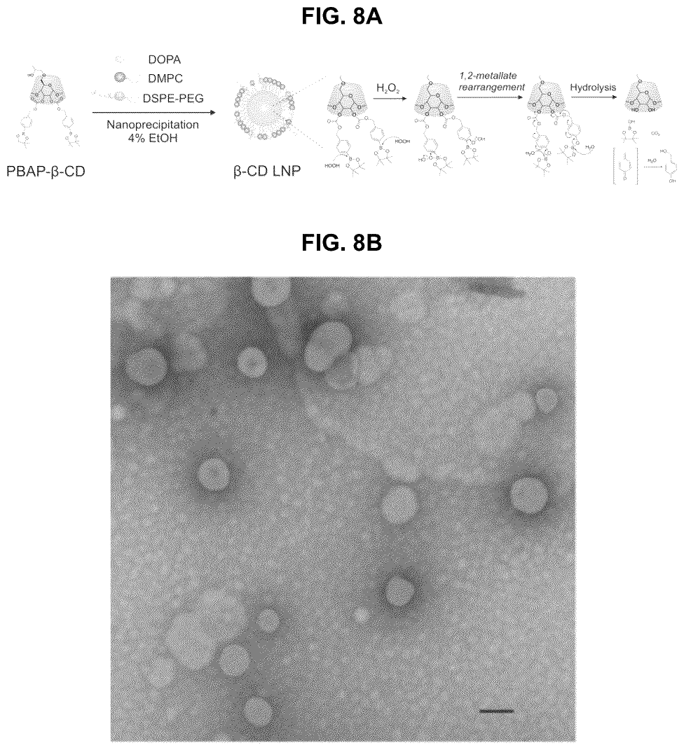

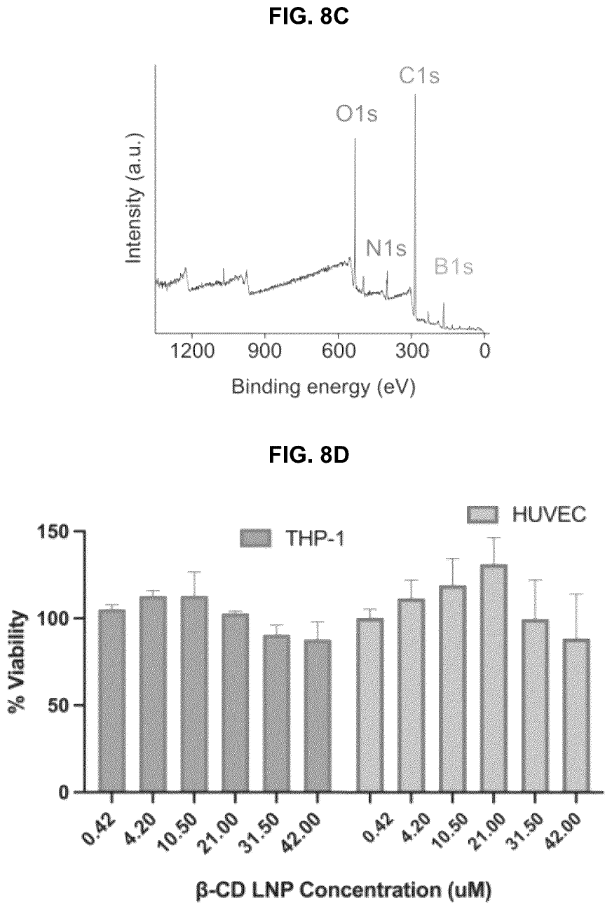

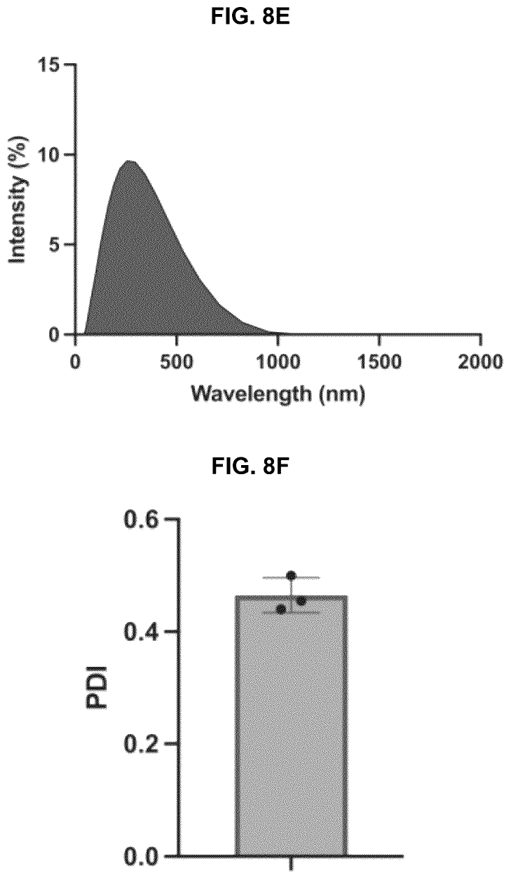

To synthesize β-CD LNPs for degrading CC, a previously reported synthesis of core-shell LNPs containing phenylboronic acid pinacol ester (PBAP)-modified β-CD cores was used (FIG. 7A). The ¹H-NMR and FT-IR spectral data confirmed the chemical composition via signature peaks (FIG. 7B-C). PBAP-modified β-CD LNPs were prepared by nanoprecipitation (FIG. 8A), resulting in an average diameter of 150 nm as visualized by transmission electron microscopy (TEM) (FIG. 8B). The synthesized PBAP was also characterized by chemical mapping via X-ray photoelectron spectroscopy (FIG. 8C), which supported the successful incorporation of PBAP-modified β-CD into the LNPs. The β-CD LNPs had low cytotoxicity in both THP-1 macrophages and human umbilical vein endothelial cells (HUVECs) (FIG. 8D), highlighting their biocompatibility in a vascular system. FIG. 8E shows dynamic light scattering (DLS) data showing the size distribution of β-CD LNPs to be between 50-1000 nm. FIG. 8F shows polydispersity index (PDI) data representing the size uniformity of β-CD LNPs.

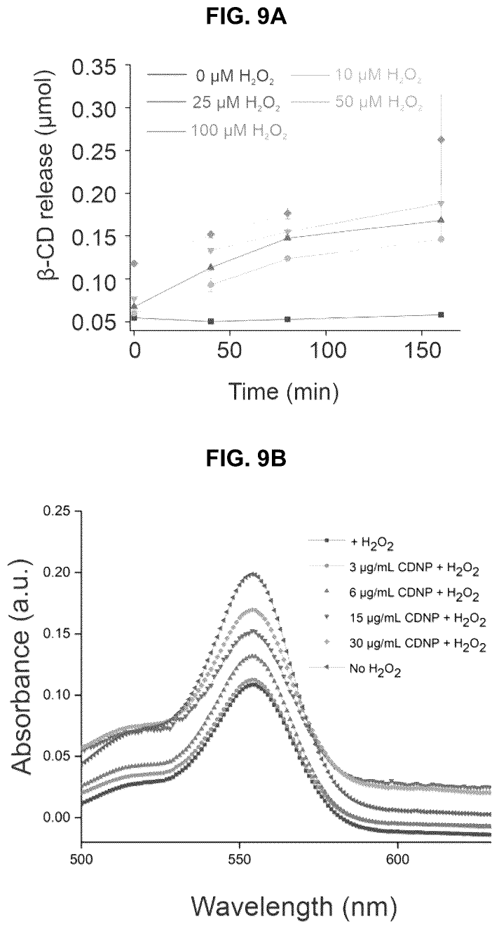

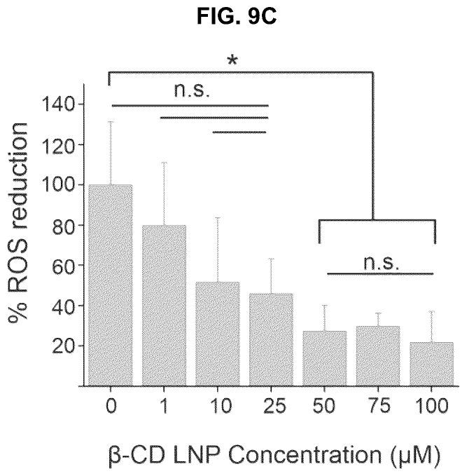

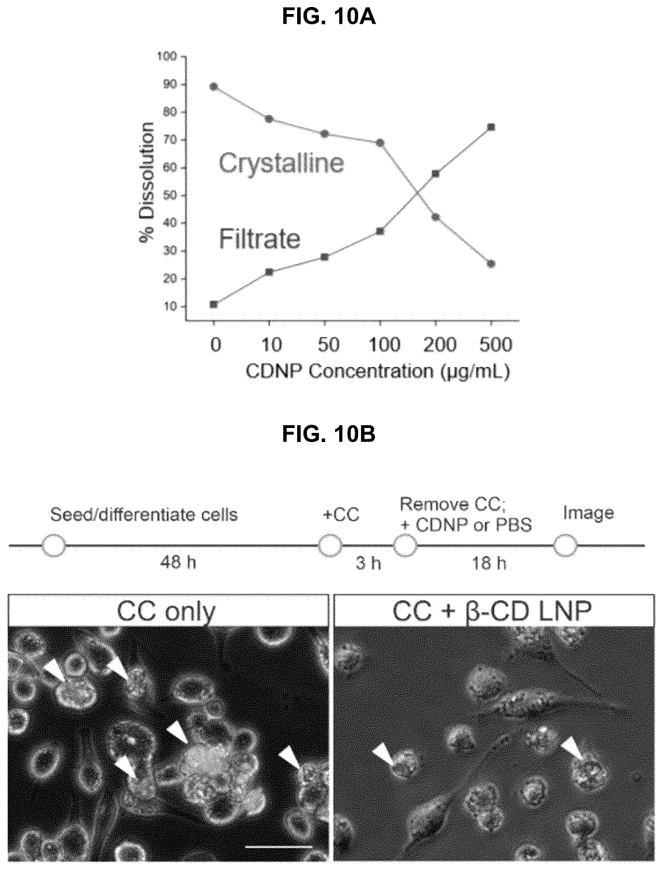

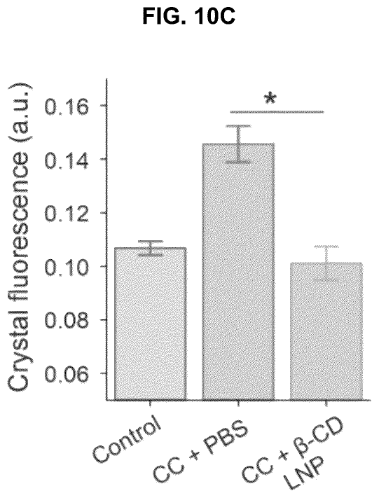

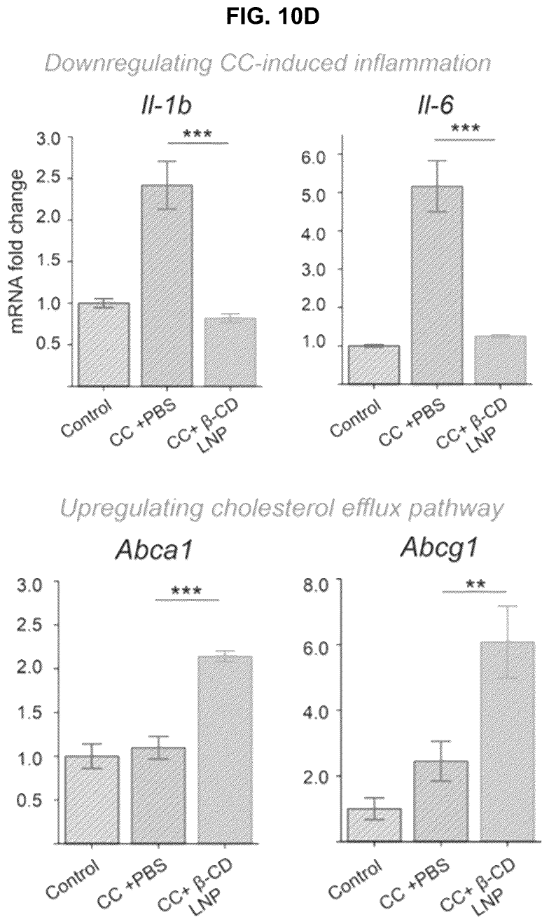

While the β-CD LNPs were highly stable under normal conditions, they readily degraded under different H₂O₂ concentrations and released HPβ-CD (FIG. 9A). The PBAP-mediated reaction products were observed to be effective at scavenging reactive oxygen species (ROS), as was demonstrated in the rhodamine quenching assay (FIG. 9B) and reducing intracellular ROS (FIG. 9C). The ROS-scavenging effect of this material has been shown to reduce inflammation significantly and improve the therapeutic outcome in atherosclerosis and ischemic stroke. Upon hydrolysis of the β-CD LNPs, the released HPβ-CD could readily form an inclusion complex with cholesterol and effectively dissolve CC in a dose-dependent manner (FIG. 10A). Furthermore, the β-CD LNP-treated macrophages harbored a lower amount of accumulated CC compared to the PBS-treated control macrophages (FIG. 10B-C). Since the ability of the β-CD LNPs to partake in the LXR pathway was not investigated previously, this property was evaluated using quantitative reverse transcription PCR (RT-qPCR). Treatment of CC-incubated THP-1 macrophages with β-CD LNPs led to a significant downregulation of the pro-inflammatory genes II-1b, Nlrp3, and 11-6 by 2.95-, 3.27-, and 4.11-fold, respectively. Furthermore, there was an upregulation of genes involved in cholesterol efflux (Abca1 and Abcg1, part of the LXR pathway) by 1.95- and 2.48-fold (FIG. 10D). A slight up-regulation of Abca1 and Abcg1 by CC was also previously reported, and β-CD could further enhance such upregulation.

### Example 3

In vitro Anchoring of β-CD LNPs onto THP-1 Monocytes and Their Protective Effects

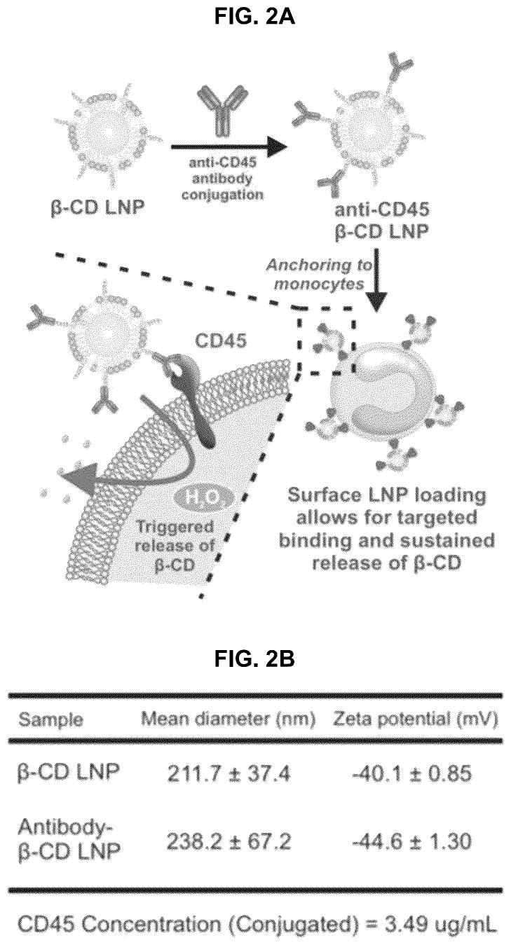

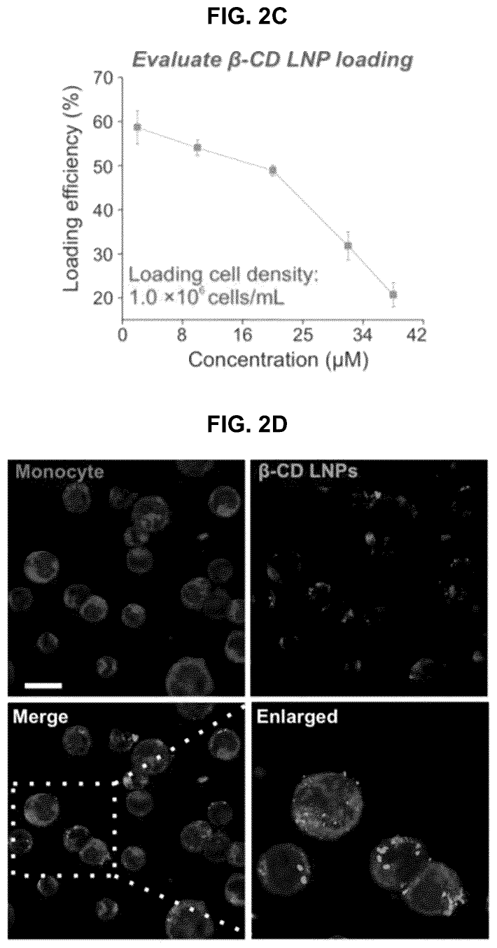

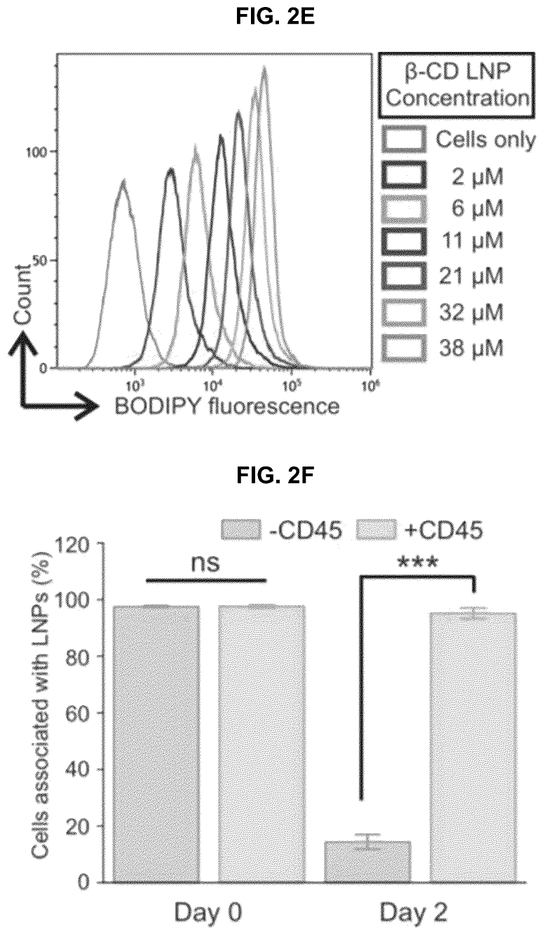

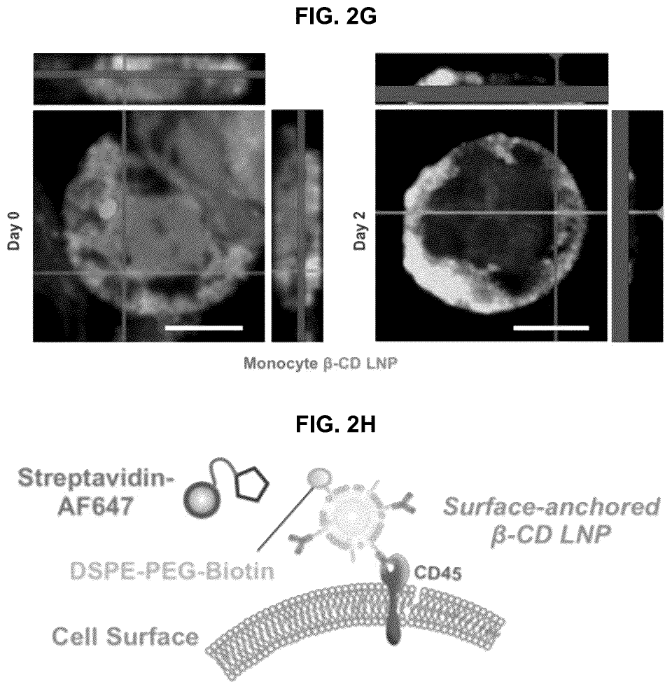

To allow controlled release of β-CD from the surface of monocytes, CD45 was chosen as the target receptor for β-CD LNP binding, since this receptor has been shown to permit surface retention of liposomes and nanogels for at least 7 days in T cells. Anti-CD45 antibodies were first coupled to the β-CD LNPs by thiol-maleimide coupling, followed by direct incubation with THP-1 monocytes to obtain β-CD LNP-bound cells (FIG. 2A). This approach enabled the triggered release of HPβ-CD by the produced H₂O₂ (intracellular or extracellular) in an inflammatory environment. DLS provided evidence of conjugation of the anti-CD45 antibodies by the increase in the hydrodynamic diameter of the β-CD LNPs after purification (FIG. 2B). It is important to note that the β-CD LNPs were prepared with a negative zeta potential to prevent any nonspecific adsorption and subsequent endocytosis, which could reduce the amount of LNPs that can be retained at the surface. This method allowed for the loading of β-CD LNPs at different concentrations with various efficiencies (FIG. 2C). Confocal microscopy revealed a robust loading of THP-1 monocytes with boron-dipyrromethene-(BODIPY-) labeled β-CD LNPs (FIG. 2D). The density of the β-CD LNPs could be tuned for optimal loading (FIG. 2E). Based on the non-toxic concentration of 38 μM (FIG. 8D), this loading concentration was chosen for subsequent experiments. Combined with the results of nanoparticle tracking analysis (NTA) (typical batch generates ˜1.93×10⁹ particles per mL), this is equivalent to ˜174 LNPs per cell. Previous studies have shown that up to 300 multilamellar liposomes could be tethered onto the surface of T cells. However, this could be due to a greater expression of CD45 on lymphocytes than on monocytes.

The targetability of CD45 was further examined, as it was previously shown for NP retention in T cells. While there was no significant difference between the control group that contained no anti-CD45 antibody versus the group that contained the anti-CD45 antibody immediately after cell backpacking, a drastic difference was observed in the amount of cell-tethered NP fluorescence after two days post-anchoring (FIG. 2F). Further, Z-stack imaging indicated the surface retention of the β-CD LNPs two days post-loading (FIG. 2G). This highlights the pivotal role of targeted binding via CD45 conjugation in increasing the surface half-life of the β-CD LNPs in leukocytes.

To further quantify the cell populations with surface β-CD LNPs over time, flow cytometry was employed to detect surface LNPs via biotin-streptavidin binding (FIG. 2H). This enabled the tracking of the number of surface NPs versus internalized NPs by examining the AlexaFluor to BODIPY fluorescence ratio. Most cells (>90%) exhibited NP on the surface after one day; however, there was a substantial decrease in the surface NP signature two days post-anchoring (FIG. 2I). This was possibly attributed to the proliferative property of THP-1 cells in the undifferentiated state (proliferation time ˜26 h) that leads to the dilution of surface-bound β-CD LNPs. A similar finding has been observed in primary T cells containing NP backpacks after stimulation in vitro.

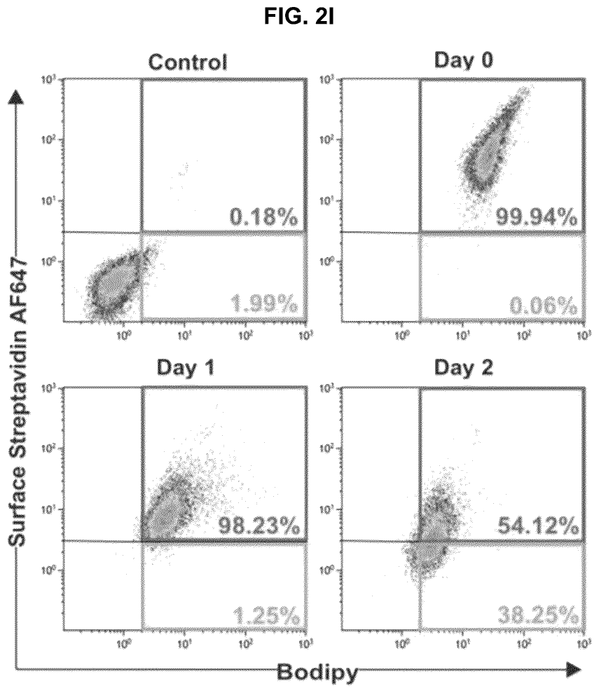

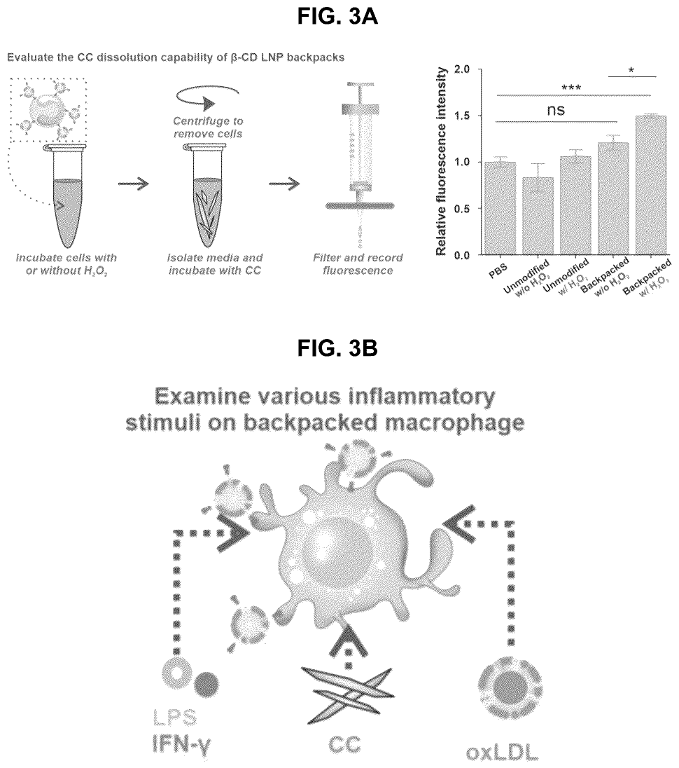

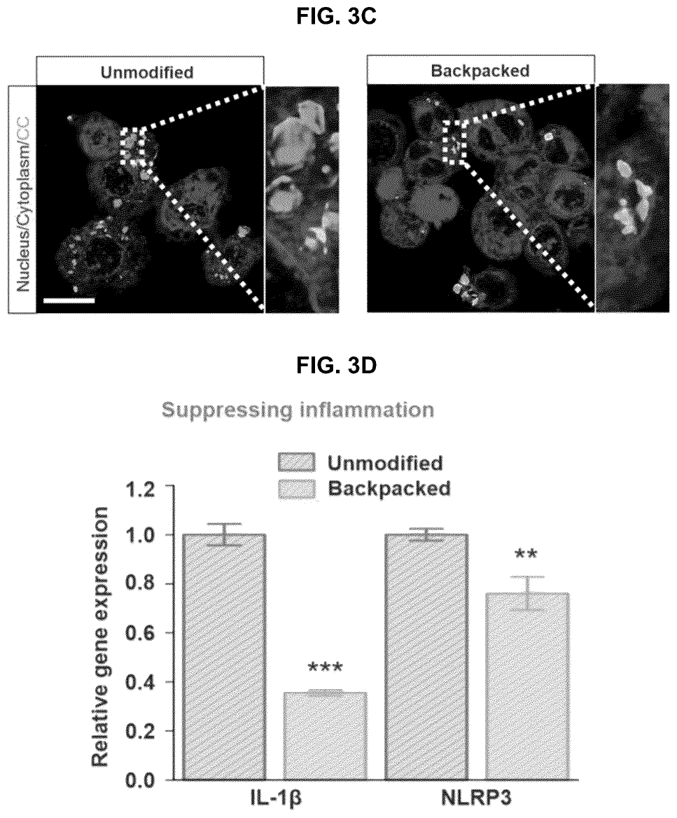

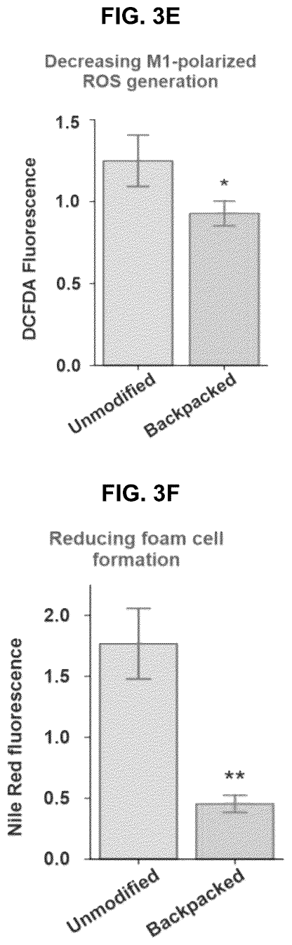

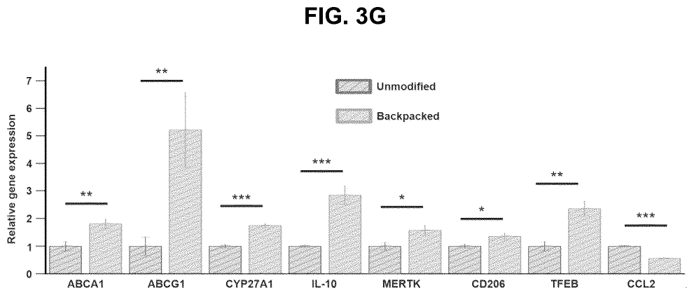

Next, the release of HPβ-CD from β-CD LNP-backpacked cells for providing sufficient concentrations in reducing CC was evaluated. THP-1 cells were pre-loaded with the LNPs, and cell supernatant was harvested at least 24 h post-loading before incubating with CC under various conditions (FIG. 3A). The HPβ-CD released from the NP-bound cells could solubilize CC in vitro, especially in those that contained H₂O₂. It was further examined whether cellular backpacking could provide any protective effects by challenging the cells with various inflammatory mediators (FIG. 3B). The decrease in intracellular CC in backpacked cells versus non-backpacked cells mirrored the results in the in vitro CC dissolution experiment (FIG. 3C). When examining the CC-induced inflammation response, a decrease was observed in gene expression of both Nlrp3 and ll-1b in THP-1 macrophages containing β-CD LNP backpacks compared to those without, supporting the protective effect of backpacking in decreasing the inflammasome response (FIG. 3D). Considering that M1 macrophages are the most common subtype detected in atherosclerotic lesions and cause plaque destabilization, control and NP-bound THP-1 macrophages were subjected to LPS and IFN-γ for 24 h after their initial differentiation for two days. It was found that control M1 macrophages had more 2′-7′-Dichlorodihydrofluorescein diacetate (DCFDA) fluorescence signal than NP-backpacked M1 macrophages, which suggested that the NP-backpacking decreased intracellular ROS levels (FIG. 3E). Similarly, a reduction in lipid accumulation in oxidized low-density lipoprotein (ox-LDL)-treated control macrophages was observed compared to the backpacked macrophages (FIG. 3F). Significantly, this further manifested the ROS and lipid scavenging advantages of surface-engineered nanoparticle backpacks.

During efferocytosis, free cholesterol from ACs contributes to the intracellular cholesterol pool, which can be toxic and leads to CC buildup. Given the multiple protective effects of β-CD LNPs, it was speculated that the β-CD LNPs could enhance efferocytosis by virtue of promoting free cholesterol conversion to oxysterols such as 25- or 27-hydroxycholesterol and activating the LXR pathway. Hence, it was examined whether the β-CD LNPs could enhance LXR signaling during efferocytosis. Upon incubation with ACs, a 1.81- and 5.22-fold up-regulation of important LXR target genes Abca1 and Abcg1 was observed; Cyp27a1, whose 27-hydroxycholesterol product is an endogenous ligand for LXR, was also increased by 1.71-fold (FIG. 3G). Previous reports have shown that HPβ-CD could facilitate cholesterol egress from lysosomes through the upregulation of LAMP-1, and that γ-CD could promote lysosomal-ER association through cellular conduits. Mechanistically, the ER-associated enzyme cholesterol 25-hydroxylase has been shown to promote efferocytosis and resolution of inflammation in mice with lung inflammation, while the failure of lysosomal acid lipase-mediated production of 25- and 27-hydroxycholesterol was shown to reduce cholesterol efflux and efferocytosis. In addition, there was a concomitant increase in Mertk and IL-10 by 1.57-fold and 2.85-fold, respectively, in β-CD LNP-containing macrophages compared with unmodified macrophages (macrophages without backpacks), supporting enhanced efferocytosis. Notably, there was also a reduction in Ccl2 expression, as well as an increase in the expression of Tfeb (autophagy marker) and Cd206 (M2 marker) expression. Collectively, these data demonstrate that β-CD LNP backpacks could reduce intracellular CC and the associated inflammation, offer protection against ROS and resistance to foam cell formation, and enhance efferocytosis while suppressing inflammation in THP-1 macrophages.

### Example 4

### CAR-M Engineering and Phagocytosis of CD47^{Hi} AC Under Normal and Inflammatory Conditions

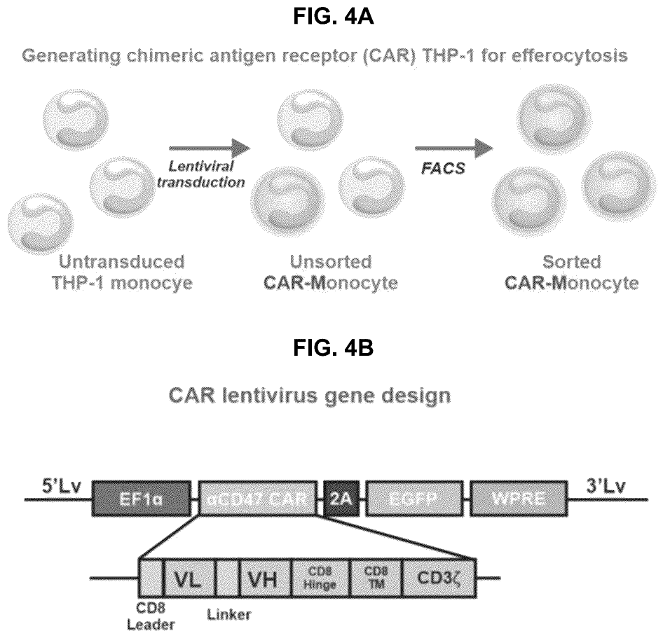

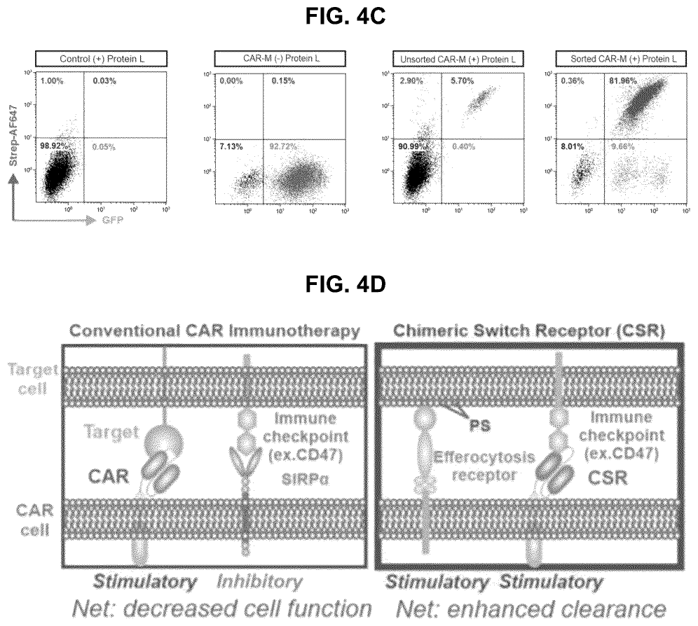

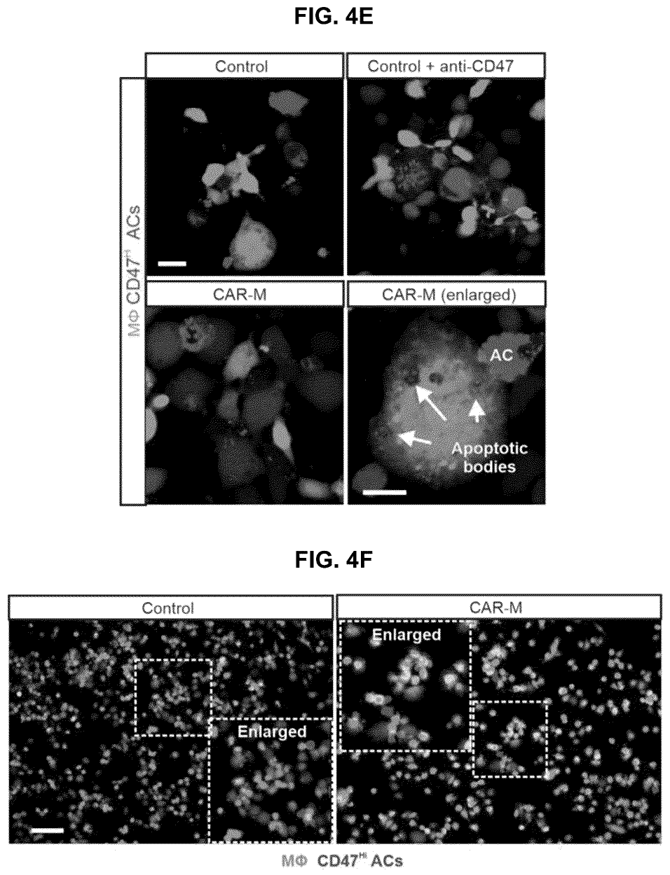

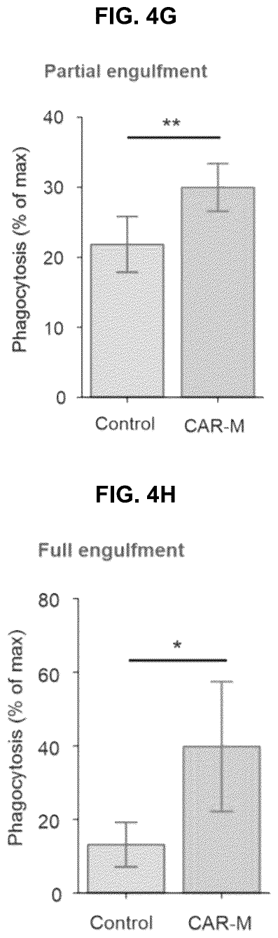

The main goal of this experimental design was to increase the LXR signaling further and the efferocytosis connection found through β-CD LNPs by manipulating the cells. A key underlying mechanism leading to defective efferocytosis in atherosclerosis is the upregulation of CD47, a myeloid immune checkpoint, in lesion smooth muscle cells. Previous works have demonstrated the pivotal role TNF-α plays in upregulating CD47 expression in various types of cells via the NF-kβ signaling pathway. This signaling event negatively impacts phagocytosis and AC clearance, leading to secondary necrosis in the atherosclerotic plaque. It was hypothesized that by engineering macrophages with anti-CD47 CAR, the phagocytosis of CD47^{Hi} ACs could be enhanced. To increase macrophage efferocytic ability against CD47^{Hi} ACs, human THP-1 monocytes were engineered with a first-generation, bicistronic lentiviral vector encoding a humanized anti-CD47 single chain antibody variable fragment (scFv), CD8 transmembrane domain, and a CD3ζ intracellular domain linked to an EGFP reporter (FIG. 4A-B). The CD3ζ domain was previously shown to trigger phagocytosis due to the presence of Immunoreceptor Tyrosine-based Activation Motifs (ITAMs). Upon lentiviral transduction and FACS, the expression of the CAR was evaluated using Protein L, which recognizes immunoglobulin light chains and scFvs. Protein L rather than CD47 was chosen for CAR detection because the cognate receptor of CD47, SIRPα, is also expressed in macrophages and thus can interfere with evaluation. Flow cytometry analysis revealed that most of the population (83.7%) were double-positive for GFP and Protein L-Alexa Fluor 647 after FACS (FIG. 4C). There was also a small population of cells (7.7%) that were GFP-positive but not stained by Protein-L, possibly due to the very low surface expression of CAR. In contrast, the unsorted batch only contained 6.8% of double-positive cells. Thus, with the dual detection of the surrogate marker (GFP) and Protein L, the surface expression of CAR in CAR-Ms could be confirmed.

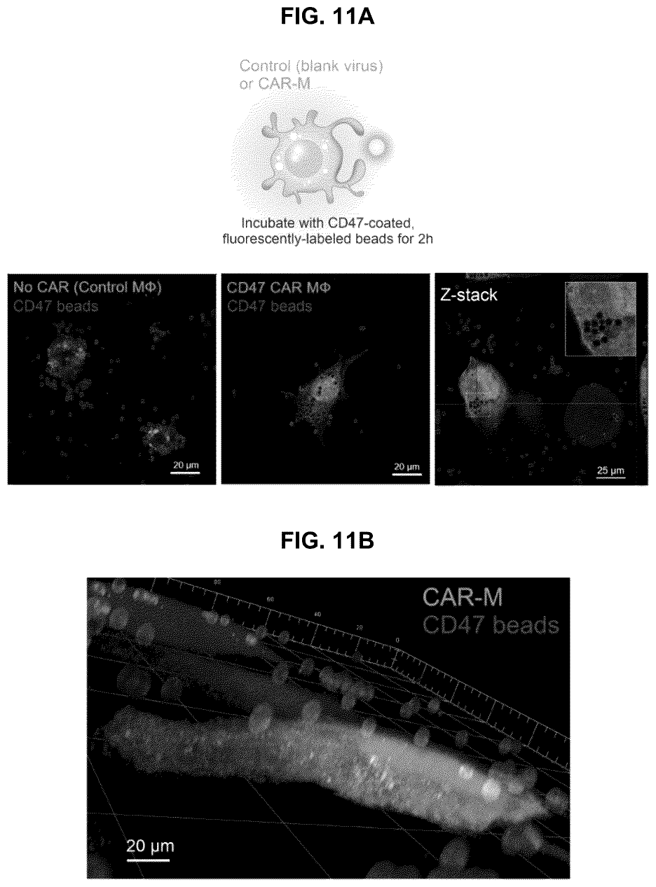

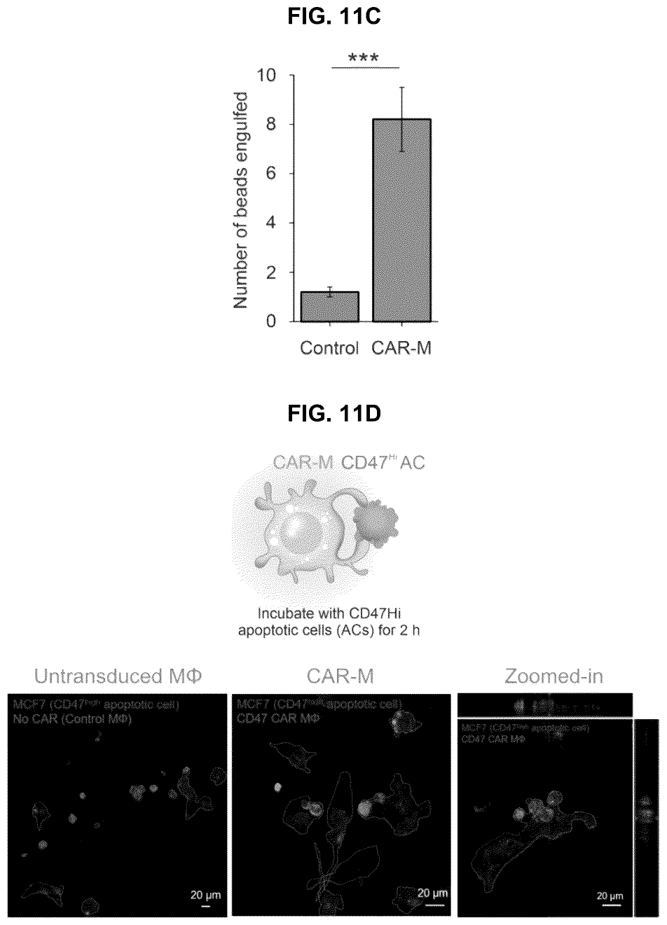

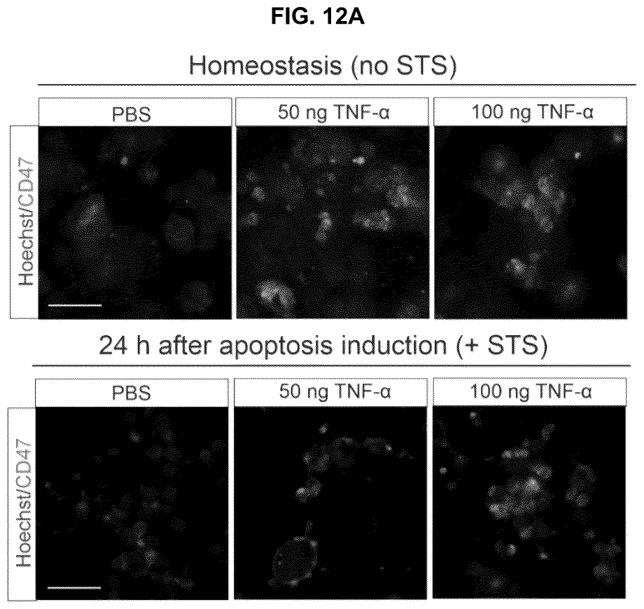

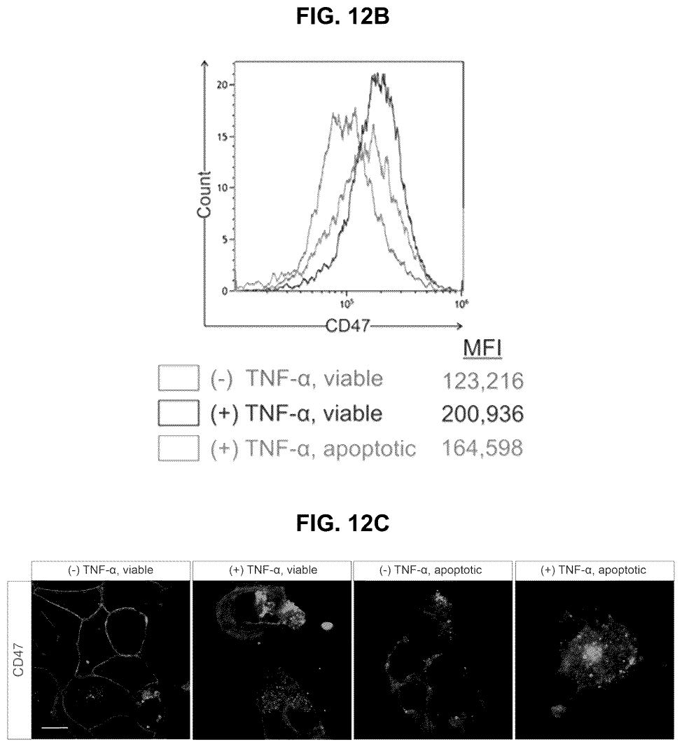

One unique aspect of the CAR design was that it permits rewiring the endogenous inhibitory response upon CD47 binding. It was speculated that the CAR-Ms could engulf the target CD47^{Hi} ACs upon binding to CD47, which normally transduces an inhibitory signal to the host phagocyte. This concept of chimeric switch receptor (CSR) has previously been demonstrated in CAR T-cells against PD-L1, but not in macrophages (FIG. 4D). To test this hypothesis, beads coated with lipids and CD47 proteins were first prepared to mimic apoptotic bodies, then bead phagocytosis assays were performed with the CAR-Ms (FIG. 11A). In contrast to the control macrophages transduced with a blank virus, the CAR-Ms took up a significantly greater number of CD47-coated beads (FIG. 11B-C). To further evaluate the phagocytic capability of CAR-Ms, MCF-7 cells were chosen as a model target due to their high basal expression of CD47. Their surface expression of CD47 could be further increased by incubation with TNF-α before subjecting the cells to apoptosis for 4 h. This allowed for the generation of phagocytosis-resistant, CD47^{Hi} early ACs that mimicked those found in chronically inflamed lesions. The upregulation of CD47 was independently verified in MCF-7 cells by fluorescence microscopy under healthy and apoptotic conditions (FIG. 12A).

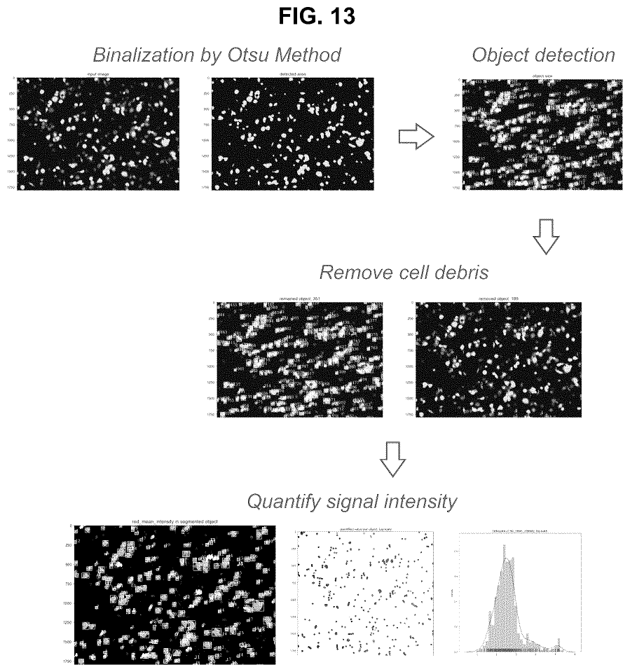

Furthermore, flow cytometry showed a blunted reduction of CD47 on the surface of TNF-α-treated ACs (FIG. 12B). Interestingly, TNF-α-treated cells not only showed increased fluorescence signal, but also exhibited more clusters (FIG. 12C). The differential clustering of CD47 under healthy versus apoptotic states was described previously and could also partially explain the increased resistance of ACs against clearance by macrophages under inflammatory conditions. Having established the target cell model, phagocytosis of CD47^{Hi} ACs was first performed with control macrophages or CAR-Ms, using anti-CD47 antibodies as a positive control. After 1 h of incubation, there was very little phagocytosis in control macrophages (FIG. 4E); in contrast, control macrophages with CD47-blocked ACs showed enhanced phagocytosis, with several whole cells in the process of being engulfed by the macrophages. When utilizing CAR-Ms, not only was the occurrence of whole-cell engulfment observed, but also the internalization of multiple cell “fragments” or apoptotic bodies. Confocal microscopy showed that the ACs were indeed internalized using Z-stack imaging (FIG. 11D). Next, the phagocytosis experiment duration was extended to 2 h, since a typical phagocytosis event is usually completed within 30-180 min. Compared to the control macrophages, there was a much greater phagocytosis of the CD47^{Hi} ACs by CAR-Ms (FIG. 4F). To further examine the phagocytosis events, the events were quantified using CellTagging, a previously reported image-based method. This enabled for the distinction of the amount of CD47^{Hi} ACs that were either partially engulfed or fully engulfed by the macrophages (FIG. 13). After 2 h of co-culture, 69.8±17.8% of CAR-Ms having engulfed one or more ACs were observed, with 39.8±17.7% of CAR-Ms having fully engulfed ACs and 30.0±3.41% of cells undergoing engulfment. On the other hand, only 34.9±9.48% of control macrophages showed engulfment, in which 13.1±6.07% of control cells had totally engulfed the target and 21.8±4.00% of cells were in the process of engulfing the ACs (FIG. 4G-I).

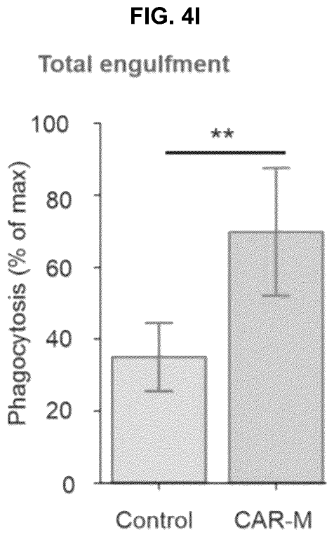

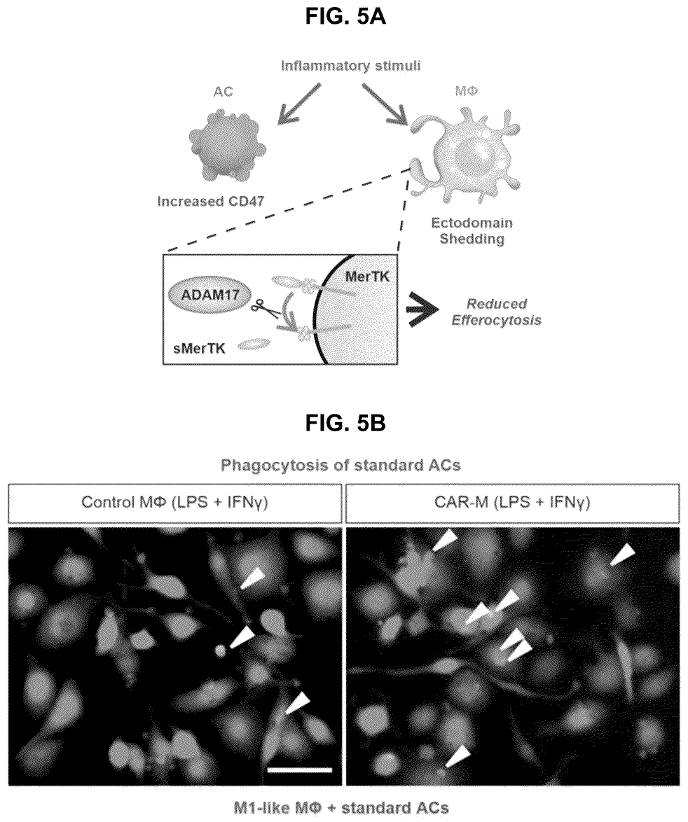

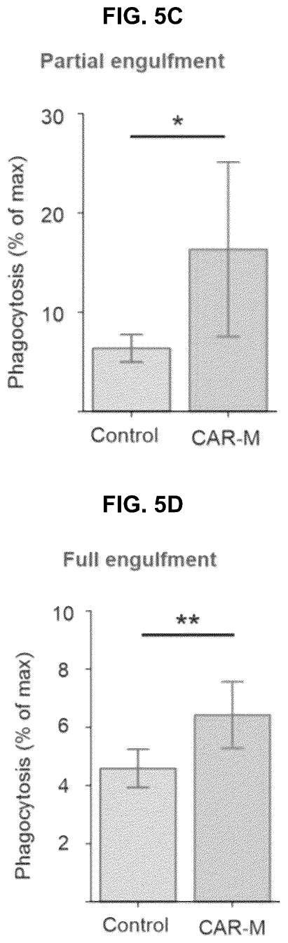

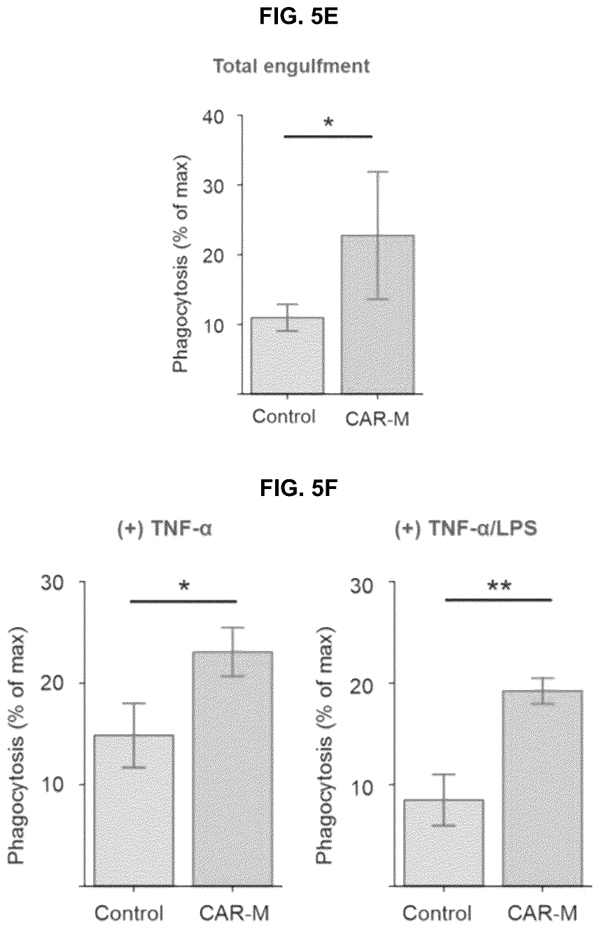

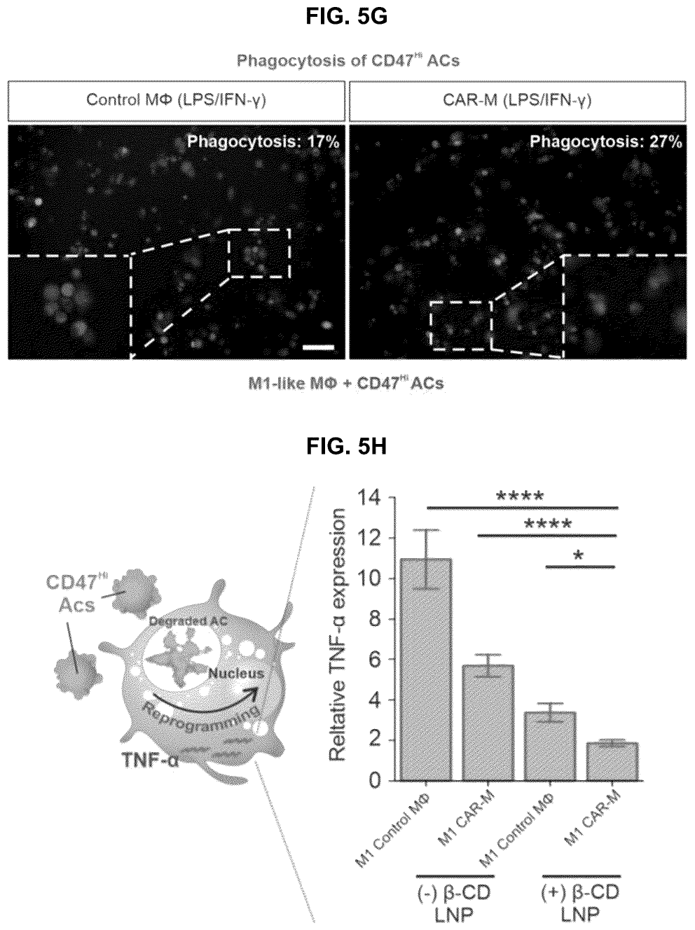

The chronic inflammatory environment negatively affects both the capacity of the macrophages to carry out phagocytosis and the ACs to be cleared (FIG. 5A). In addition to the overexpression of CD47 on ACs, lesion macrophages also suffer reduced efferocytic capability due to ectodomain shedding of the tyrosine kinase receptors (i.e., TAM receptors, Tyro3, Axl, and MerTK) mediated by metalloproteinases such asA Disintegrin and Metalloprotease 17 (ADAM17), the expression of which can be upregulated with LPS. Having demonstrated the potential of anti-CD47 CARs in enhancing the phagocytosis of CD47^{Hi} ACs, it was hypothesized that the anti-CD47 CARs could also serve as alternative receptors to rescue efferocytosis because of the loss in binding affinity between host SIRPα and CD47 on the surface of ACs.

To test this hypothesis, both the control macrophages and CAR-M were subjected to various types of inflammatory signals without perturbing the levels of CD47 on ACs. The inflammatory environment of the lesion was first simulated by incubating phagocytes with LPS and IFN-γ to induce M1 polarization. Standard ACs were subsequently prepared from Jurkat cells (i.e., without inducing elevated CD47 expression) using established methods for co-culture experiments. It was observed that while both M1 control macrophages and CAR-Ms exhibited reduced phagocytosis, CAR-Ms could phagocytose more JurkatACs (FIG. 5B). Further analysis revealed 6.42±1.15% and 16.3±8.81% of M1 CAR-M partaking in full and partial engulfment, respectively, compared to 4.59±0.66% and 6.37±1.39% of M1 control macrophages engaged in such processes (FIG. 5C-E). Similarly, while control macrophages and CAR-Ms exposed to either TNF-α alone or TNF-α and LPS exhibited reduced phagocytosis, CAR-Ms outperformed control macrophages in phagocytosis (23.1±2.40% for TNF-α alone and 19.2±1.27% for TNF-α and LPS in CAR-M; 14.8±3.19% for TNF-α alone and 8.47±2.52% for TNF-α and LPS in control macrophages) (FIG. 5F).

To further examine the full extent of phagocytosis under inflammatory conditions, M1 CAR-M versus M1 control macrophage-mediated phagocytosis of CD47^{Hi} ACs was evaluated, since CAR-Ms would be subjected to a similar inflammatory onslaught as endogenous lesion macrophages. Fluorescence microscopy showed ˜27% of M1 CAR-Ms having engulfed CD47^{Hi} ACs, compared to ˜17% in control macrophages in 2 h (FIG. 5G). Furthermore, RT-qPCR analysis showed the most significant reduction of TNF-α in the M1 CAR-M/β-CD LNP group compared to other conditions (FIG. 5H). This result also indirectly confirmed the timely phagocytosis of ACs, since phagocytosis of necrotic cells leads to increased production of TNF-α. Together, these data indicated that anti-CD47 CARs not only exert improved phagocytosis of hard-to-clear CD47^{Hi} ACs in CAR-M but could also serve as alternative receptors to induce target cell engulfment in different types of ACs.

### Example 5

Combining β-CD LNPs with CAR-M for Enhanced Phagocytosis and Transmigration in a Microfabricated System

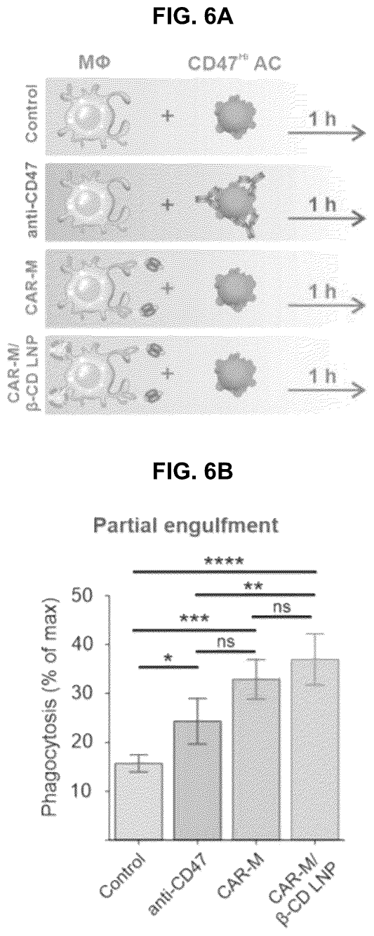

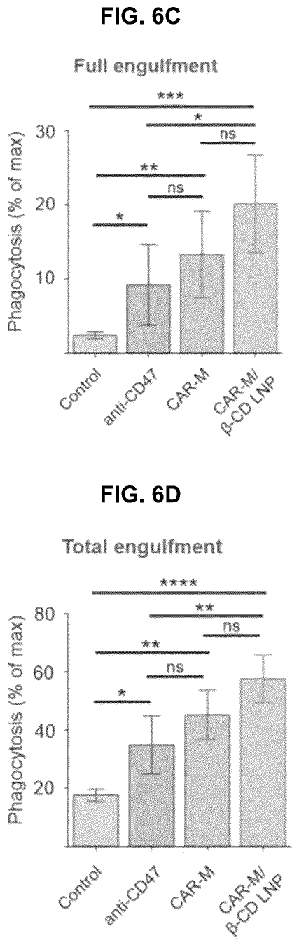

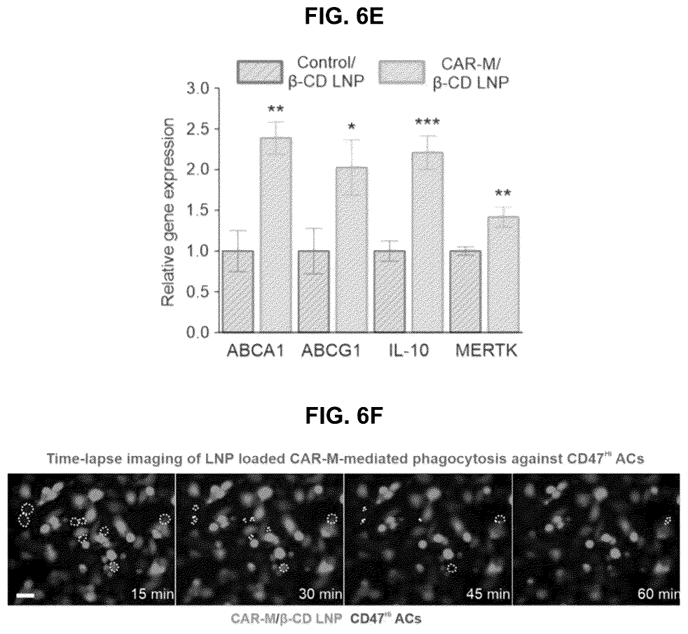

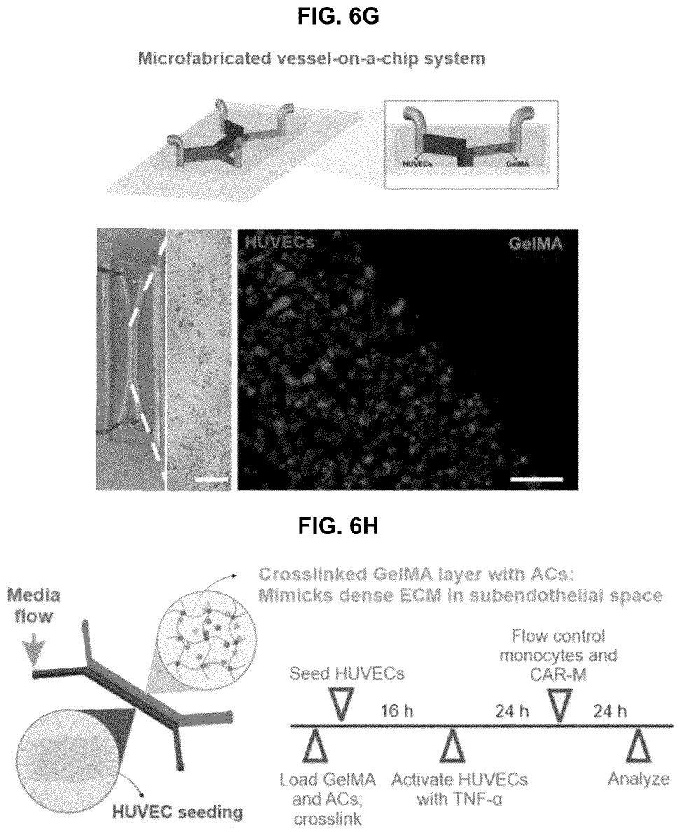

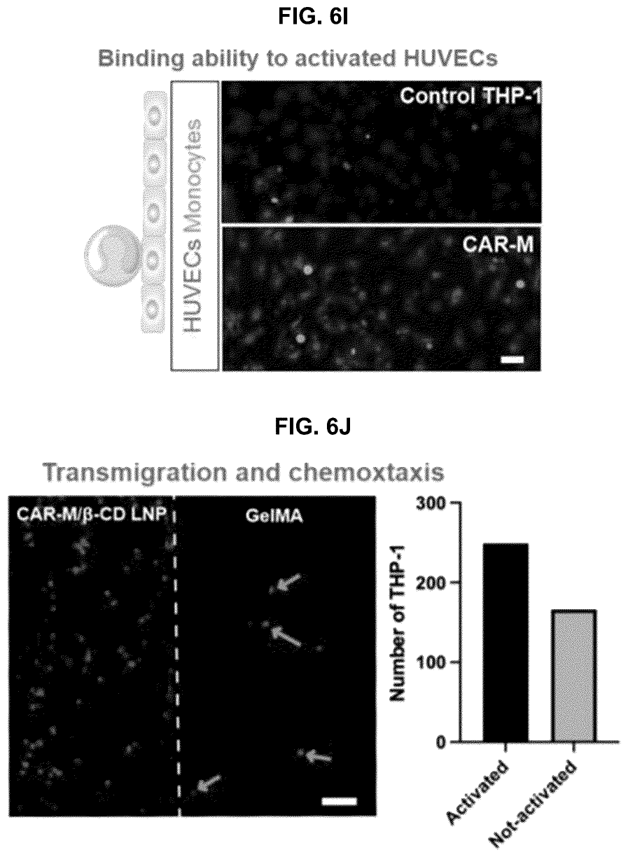

To better understand how CAR-Ms compare with the standard regimen of CD47 blockade, and whether β-CD LNPs could enhance the phagocytosis activity, the degree of total, full, and partial phagocytosis of CD47^{Hi} ACs was investigated (FIG. 6A). Quantification analysis revealed increased phagocytosis in both control macrophages treated with anti-CD47 antibodies, CAR-Ms, and CAR-Ms/β-CD LNPs (FIG. 6B-D. Although there was no statistically significant difference in total engulfment between the CAR-M versus CD47 blockade group (p=0.29), there was significantly greater phagocytosis in CAR-M/β-CD LNPs compared to control macrophages with anti-CD47 antibodies (p=0.0071), implying some synergy between CAR-mediated phagocytosis and the HPβ-CD released from LNPs. This enhancement mirrors the increase in anti-inflammatory cytokine expression after efferocytosis results from the in vitro β-CD LNP backpack experiments (FIG. 4H). To confirm that the enhancing effect was from the engineered CAR, RT-qPCR of control macrophages and CAR-Ms loaded with β-CD LNPs was performed after phagocytosis of CD47^{Hi} ACs. An increased expression of the LXR target genes Abca1 and Abcg1 was observed in CAR-Ms by 2.39- and 2.03-fold, respectively. Furthermore, Mertk and IL-10 levels increased by 1.42- and 2.21-fold compared to control macrophages with β-CD LNPs (FIG. 6E). Live imaging further confirmed the timely reduction of ACs by CAR-M/β-CD LNP within 1 h (FIG. 6F). Together, these findings provided evidence for a synergistic enhancement of efferocytosis by dual nanoparticle and CAR engineering.

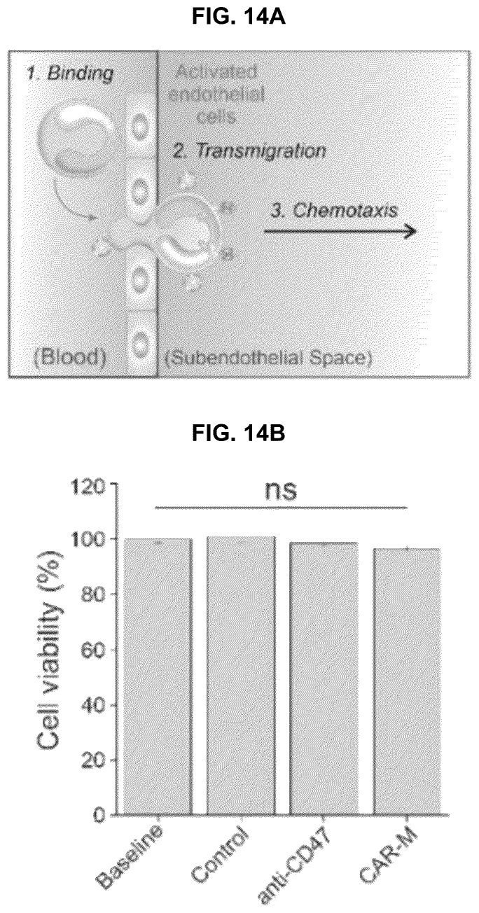

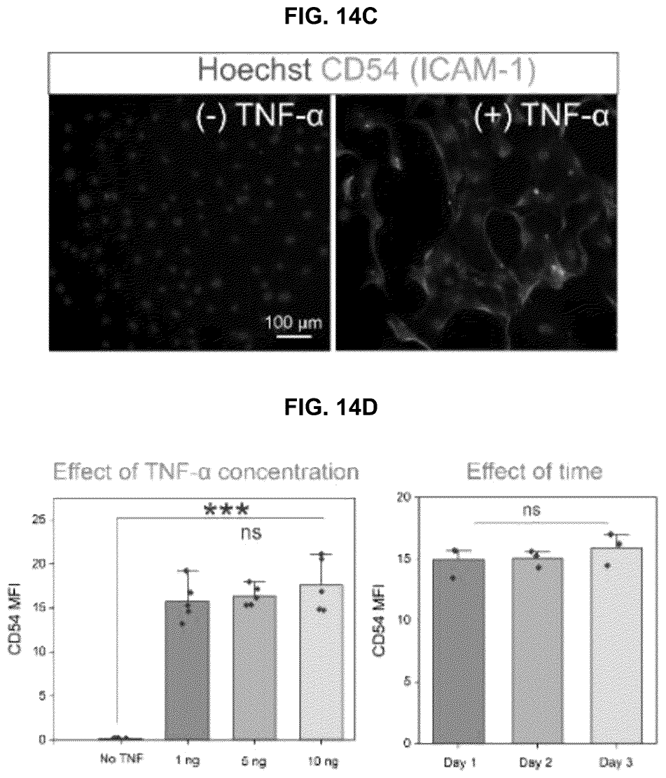

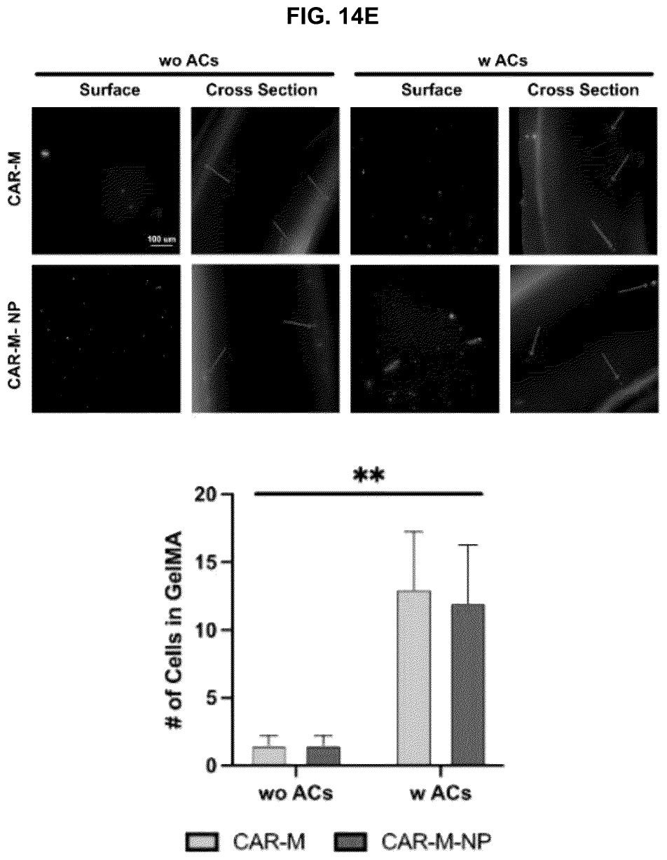

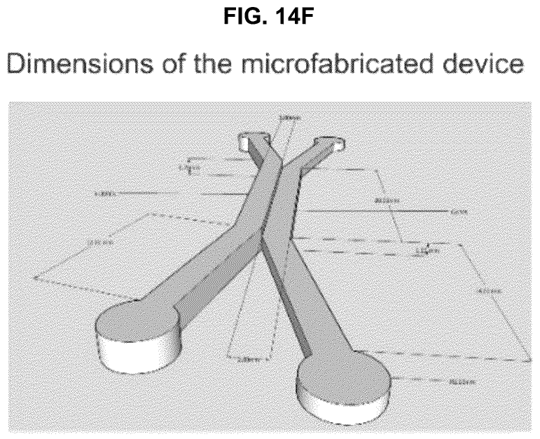

One key advantage of a cell-based therapy platform is the homing ability of cells to target tissues. Monocytes have the ability to travel to atherosclerosis-affected areas by binding to activated endothelial cells and then passing through the endothelial layer into the subendothelial layer (diapedesis), as illustrated in (FIG. 14A). To demonstrate the feasibility of CAR-Ms as a potential therapeutic platform in a vascular system, the cytotoxicity of CAR-Ms was first tested by performing co-culturing experiments with HUVECs. A 50/50 culture media was used for the greatest viability of THP-1 cells and HUVECs. This resulted in a ˜14% reduction in the cell viability of HUVECs. Considering this, no observable toxicity was associated with HUVECs co-incubated with CAR-Ms after 24 h (FIG. 14B).

Having confirmed the non-toxic nature of CAR-Ms, the conditions for optimal HUVEC activation for cell binding was next evaluated by treating the cells with TNF-α at different dosages for different time periods (FIG. 14C-D). The subendothelial space, or arterial intima, is heavily comprised of extracellular matrix materials, such as collagen, fibronectin, and laminin. For the CAR-Ms to function as intended, cell penetration and migration studies were performed using gelatin-methacrylate (GeIMA) hydrogels. Initial attempts using 10% cross-linked GeIMA did not result in meaningful penetration or migration. Adjusting the cross-linked concentration to 6% and adding ACs resulted in more significant cell migration (FIG. 14E). With that, a microfluidic device was constructed to examine the adhesion and trans-endothelial migration of CAR-Ms (FIG. 6G) (FIG. 14F). The device was constructed with one pathway to allow for the seeding and adhesion of HUVECs, and a second channel that was filled with GeIMA containing ACs, replicating the subendothelial space of atherosclerotic plaque tissue. Next, an optimized procedure for cell seeding, hydrogel loading, and cell activation was developed (FIG. 6H). Upon 24 h of co-incubation, the binding of CAR-Ms with the activated HUVECs could be readily observed, as similarly shown in control THP-1 monocytes (FIG. 6I). To demonstrate the potential of CAR-Ms in acting as therapeutic vehicles, β-CD LNP backpacks were anchored onto the cells and diapedesis was examined. Confocal microscopy revealed an increased number of CAR-Ms transmigrated to the GeIMA layer in the activated HUVEC group compared to the non-activated group, demonstrating the feasibility of the LNP-loaded CAR-Ms as a platform for targeting inflamed atherosclerotic lesions in vivo (FIG. 6J).

Given that macrophages play a profound role in the catabolic turnover of cellular materials in peripheral tissues, an objective of these studies was to enhance the capabilities of macrophages to clear ACs that overexpress CD47 because of chronic inflammation. To achieve this, a new type of CAR-Ms called chimeric switch receptor macrophages (CSR-Ms) were developed. This approach combined the anti-CD47 scFv with a stimulatory intracellular signaling domain, effectively reversing the inhibitory signaling linked to the SIRPα-CD47 axis. The phagocytosis activity of the developed CAR-Ms was significantly more potent and enhanced compared to standard control macrophages, both in standard and inflammatory settings. Moreover, the CAR-Ms were able to target ACs with normal or elevated expression of CD47.

Furthermore, to augment macrophage function, including efferocytosis, LNPs comprised of PBAP-modified β-CD were used. Anchoring the LNPs as cellular backpacks on the CAR-Ms may improve the bioavailability of β-CD in vivo. This potential was demonstrated in a blood vessel-on-α-chip model, where CAR-Ms could bind to and transmigrate into the hydrogel layer, mimicking the intima while carrying the LNPs. Previous studies have attributed the beneficial effects of these LNPs to the suppression of ROS. Indeed, oxidative stress has been shown to negatively impact cholesterol efflux and efferocytosis.

The results of the study described herein reveal that the beneficial effects of the disclosed approach are due in part to the release of HPβ-CD, which promotes metabolic reprogramming and improves LXR signaling. Previous works have shown that the LXR pathway is important in maintaining cholesterol homeostasis and reducing inflammation. Increased catabolism by HPβ-CD released from the LNPs can reduce the overwhelming cholesterol burden caused by AC ingestion through metabolic reprogramming. The results described herein show that the combination of HPβ-CD and anti-CD47 CAR works synergistically to enhance phagocytosis, surpassing the effects of CD47 blockade alone. To further improve the efficacy of this approach, various CAR iterations will be developed having additional activation domains or linked intracellular signaling domains to increase the release of anti-inflammatory cytokines, which could improve efferocytosis.

These studies demonstrate the successful development of a variation of CAR-M, namely CSR-M, which considerably improves macrophage phagocytic activity of its related target, apoptotic cells (ACs). These studies further demonstrate that using LNPs comprised of PBAP-modified β-CD with the CAR-Ms could enhance macrophage function. Moreover, the disclosed technology allows for a faster and more effective way to clear CD47^{Hi} ACs, providing a wider clearance window and decreasing the chances of secondary necrosis in the context of atherosclerosis.

What is claimed:

1. A modified macrophage or monocyte comprising:
surface-expressed CD47-targeted chimeric antigen receptor (CAR) proteins; and
lipid-based particles conjugated to a surface of the modified macrophage or monocyte, wherein the lipid-based particles comprise a β-cyclodextrin (β-CD).

2. The modified macrophage or monocyte of claim 1, wherein the modified macrophage or monocyte is derived from a primary macrophage or monocyte, or wherein the modified macrophage or monocyte is derived from an induced pluripotent stem cell (iPSC).

3. The modified macrophage or monocyte of claim 1, wherein the CD47-targeted CAR proteins comprise an anti-CD47 single-chain variable fragment (scFv) comprising VL and VH; a CD8 hinge domain; a CD8 transmembrane domain; and a CD3ζ signaling domain.

4. The modified macrophage or monocyte of claim 1, wherein the CD47-targeted CAR proteins comprise an amino acid sequence having at least 90-99% identity to SEQ ID NO: 1.

5. The modified macrophage or monocyte of claim 4, wherein the CD47-targeted CAR proteins comprise an amino acid sequence of SEQ ID NO: 1.

6. The modified macrophage or monocyte of claim 1, wherein the lipid-based particles are lipid nanoparticles (LNPs) or liposomes.

7. The modified macrophage or monocyte of claim 6, wherein the lipid-based particles are LNPs comprising one or more of 1,2-dimyristoyl-sn-glycero-3-phosphocholine (DMPC), 1,2-dioleoyl-sn-glycero-3-phosphate (DOPA), cholesterol, or 1,2-distearoyl-sn-glycero-3-phosphoethanolamine-N-[amino(polyethylene glycol)-2000](DSPE-PEG(2000)).

8. The modified macrophage or monocyte of claim 1, wherein the β-CD is hydroxypropyl β-CD (HPβ-CD).

9. The modified macrophage or monocyte of claim 1, wherein the β-CD is modified with phenylboronic acid pinacol ester (PBAP).

10. The modified macrophage or monocyte of claim 1, wherein the lipid-based particles comprise a surface-conjugated anti-CD45 antibody that binds to CD45 expressed on the surface of the modified macrophage or monocyte.

11. The modified macrophage or monocyte of claim 1, wherein the lipid-based particles have a mean diameter of about 100 nm to about 350 nm.

12. The modified macrophage or monocyte of claim 1, wherein the lipid-based particles have a negative zeta potential of about −35 mV to about −50 mV.

13. The modified macrophage or monocyte of claim 1, wherein about 50 lipid-based particles to about 300 lipid-based particles are conjugated to the surface of the modified macrophage or monocyte.

14. The modified macrophage or monocyte of claim 13, wherein about 100 lipid-based particles to about 200 lipid-based particles are conjugated to the surface of the modified macrophage or monocyte.

15. The modified macrophage or monocyte of claim 1, wherein the modified macrophage or monocyte has enhanced phagocytosis and transmigration properties.

16. A pharmaceutical composition comprising the modified macrophage or monocyte of claim 1.

17. A method of treating atherosclerosis in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

18. The method of claim 17, wherein the β-CD is released from the lipid-based particles in response to elevated levels of reactive oxygen species (ROS) in the subject.

19. The method of claim 17, wherein the modified macrophage or monocyte reduces an amount of CD47-overexpressing apoptotic cells in the subject.

20. The method of claim 19, wherein the CD47-overexpressing apoptotic cells are apoptotic foam cells.

21. The method of claim 17, wherein the modified macrophage or monocyte reduces an amount of insoluble cholesterol in the subject.

22. The method of claim 17, wherein the modified macrophage or monocyte reduces inflammation in the subject.

23. A method of treating cardiovascular disease in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

24. A method of treating inflammation in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

25. A method of treating a chronic inflammatory disease in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

26. A method of treating a wound in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).

27. A method of treating a spinal cord injury in a subject, the method comprising administering to the subject a therapeutically effective amount of a pharmaceutical composition comprising a modified macrophage or monocyte comprising: surface-expressed CD47-targeted chimeric antigen receptor (CAR) proteins; and lipid-based particles conjugated to a surface of the modified macrophage or monocyte, wherein the lipid-based particles comprise a β-cyclodextrin (β-CD).
